(19) United States
(12) Patent Application Publication
(10) Pub. No.: US 2005/0222418 A1
(43) Pub. Date: Oct. 6, 2005

(54) Novel tyloindicines and related processes, pharmaceutical compositions and methods

(76) Inventors: David C. Baker, Knoxville, TN (US); Yung-Chi Cheng, Woodbridge, CT (US); Sanbao Zhong, Highland Park, NJ (US)

Correspondence Address: Henry D Coleman;Coleman Sudol Sapone, 714 Colorado Avenue, Bridgeport, CT, 06605-1601, US

(21) Appl. No.: 10/502,074

(22) PCT Filed: Feb. 12, 2003
(86) PCT No.: PCT/US03/04072

§ 371 Date: Aug. 19, 2004

Related U.S. Application Data
(60) Provisional application No. 60/357,354, filed on Feb. 15, 2002.

(51) Int. Cl. A61K 31/4745; C07D 455/02; C07F 7/02

(52) U.S. Cl. 546/138; 514/306; 546/14

(57) ABSTRACT

The invention provides novel tyloindicines analogues and related processes, pharmaceutical compositions, and methods. The novel tyloindicines are useful in a wide variety of antiviral, antineoplastic, antibacterial, and anti-inflammatory applications. Preferred embodiments of the instant invention include the novel tyloindicine designated herein as compound IT-3 (NSC-716802). Compounds of the instant invention have exhibited potent antiviral and anticancer activity in vitro. The invention further provides novel methods of treating neoplastic, bacterial, viral, and anti-inflammatory disorders using tyloindicines including the novel tyloindicine analogues of the instant invention. The invention also provides novel syntheses of tyloindicines including the novel tyloindicines analogues of the instant invention.

### FIELD OF THE INVENTION

The invention provides novel tyloindicine analogues and related processes, pharmaceutical compositions, and methods. The novel tyloindicines are useful in a wide variety of antiviral, antineoplastic, antibacterial, and anti-inflammatory applications. Preferred embodiments of the instant invention include the novel tyloindicine analogues designated herein as compounds II-2 (DCB-3501, NSC-717335) and II-3 (DCB-3503, NSC-716802 or ZH-152). Compounds of the instant invention have exhibited potent antiviral and anticancer activity in vitro. The invention further provides novel methods of treating neoplastic, bacterial, viral, and inflammatory disorders using tyloindicines, including the novel tyloindicine analogues of the instant invention. The invention also provides novel syntheses of tyloindicines, including syntheses of the novel tyloindicine analogues of the instant invention.

### BACKGROUND OF THE INVENTION

Notwithstanding the progress that has been made to date in identifying compositions that have either anticancer, antiviral, antibacterial, or anti-inflammatory activity, the need continues to exist for biologically active compositions that exhibit a wide range of such properties. In particular, there is a need for compositions that ideally exhibit some level of activity against all such disorders. Such compositions must be safe and well-tolerated and be suitable for use in numerous pharmaceutical dosage forms and routes of administration. Preferably, the compositions would prove active against neoplastic, viral, bacterial, and inflammatory disorders upon administration to a patient in need, and would also be useful in treating bacterial infections such as tuberculosis-associated viral infections such as AIDS. There is a particular need for compounds useful in treating drug-resistant cancers.

Until now, the potential of tyloindicines to satisfy broadly the aforementioned needs has remained uncertain and, essentially, undeveloped. Further, until now, tyloindicines have proven very difficult to synthesize.

Tyloindicines (also referred to herein as “tylos”) such as tyloindicines F, G, H, and I (tylo F, tylo G, tylo H, and tylo I) belong to a group of alkaloids that have been isolated from Tylophora indica, a plant native to India. Ali, M.; et al., Tylophora indica. Phytochemistry 1989, 28, 3513-3517. Tylos F and G have a tertiary hydroxyl group on the indolizidine moiety. This group of compounds has been available in only limited quantities from the natural source and are presently unavailable for further research, due in part to their low yields from the plant: 0.004% and 0.001%, respectively.

While there has been synthetic work carried out in the general area of tylophora (indolizidine) alkaloids, synthesis of these potent (especially the hydroxylated) compounds in optically active form has remained elusive. Faber, L.; et al., Stereospecific synthesis of a 9,11,12,13,13a,14-hexahydrodibenzo(f,h)-pyrrolo(1,2-[b]isoquinoline alkaloid. Helv. Chim. Acta 1973, 56, 2882-2884; 7) Comins, D. L.; Chem, X.; Morgan, L. A. Enantiopure N-acyldihydropyridones as synthetic intermediates: Asymmetric synthesis of -septicine and -tylophorine. J. Org. Chem. 1997, 62, 7435-7438.

The present invention has been supported by one or more government grants funded by the National Institutes of Health. As such, the government retains certain rights in the invention.

### OBJECTS OF THE INVENTION

It is an object of the instant invention to provide novel, biologically active tyloindicine analogues that prove active against a wide range of neoplastic and inflammatory disorders or as a treatment for Epstein-Barr virus (EBV) infections or EBV-related lymphoma or cancer.

It is a further object of the instant invention to provide novel, biologically active tyloindicine analogues that may be employed in anticancer and anti-inflammatory pharmaceutical compositions or as anti-EBV infections or in conditions which appear secondary to EBV infections, such as EBV-related lymphoma or cancer.

It is a further object of the instant invention to provide novel, biologically active tyloindicine analogues that are safe and well-tolerated.

It is a further object of the instant invention to provide methods of using tyloindicines, including novel, biologically active tyloindicine analogues of the instant invention, to treat neoplastic and inflammatory disorders, as well as EBV infections or conditions which appear secondary to EBV infections, such as EBV-related lymphoma or cancer.

It is a further object of the instant invention to provide novel processes for making tyloindicines, including the novel, biologically active tyloindicine analogues.

It is a further object of the instant invention to provide novel, biologically active tyloindicine analogues useful in treating drug resistant cancers.

### SUMMARY OF THE INVENTION

In accordance with the above stated objects, the instant invention provides novel tyloindicine analogues according the general formula (A): 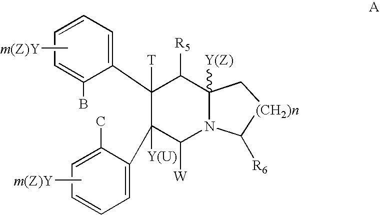
Wherein Y is O, S, NH, CH₂ or is absent;
Each (Z) is independently H, a (C₁-C₄) alkyl, a substituted alkyl, an aryl, a substituted aryl, alkyl silyl, a heterocycle, a substituted heterocycle, with the proviso that not all Z are H when
Y is absent;
(U) is H, a (C₁-C₄) alkyl, a substituted alkyl, an aryl, a substituted aryl, alkyl silyl, a heterocycle, a substituted heterocycle, or together with W forms a double bond in the nitrogen containing ring or together with T forms a double bond in the nitrogen containing ring;
T is H, forms a double bond with the carbon to which R₅ is attached or forms a double bond with the carbon attached to Y(U);
W is H or forms a double bond with the carbon attached to Y(U) in the nitrogen containing ring;
R₅ is H, OH, ═O (to form a carbonyl group with the carbon to which it is attached), a carboxyl (carboxylate group), —OC(O)Rₓ group, a —C(O)Rₓ, or a —C(O)ORₓ group, where Rₓ is a C₂ to C₁₅ alkyl, preferably a C₂ to C₈ alkyl;
R₆ is H, OH, ═O (to form a carbonyl with the carbon to which it is attached), a carboxyl (carboxylate group), a —OC(O)Rₓ group, a —C(O)Rₓ, or a —C(O)ORₓ group, where Rₓ is defined above;
B is Y(Z) or together with C forms a bond between the two phenyl rings to which each of B and C is attached;
C is Y(Z) or together with B forms a bond between the two phenyl rings to which each of B and C is attached;
m is from 0 to 4, preferably 1 or 2;
n is from 0 to 3, preferably 1 or 2;
   and epimers, pharmaceutically acceptable salts, solvates or polymorphs thereof.

In accordance with certain preferred embodiments according to the present invention, the invention provides compounds of the formulae (I) and (II): 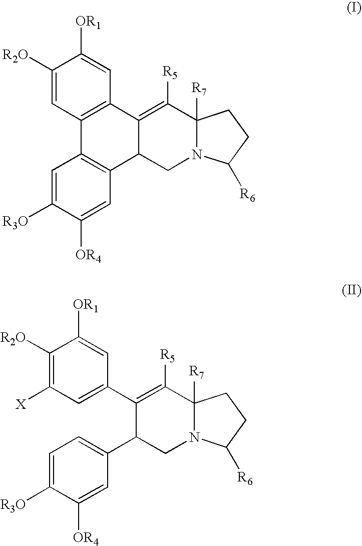
and epimers, pharmaceutically acceptable salts, solvates, or polymorphs thereof,
wherein: R₁, R₂, R₃, R₄ and R₇ are independently H, a C₁-C₄ alkyl, a substituted alkyl, an aryl, a substituted aryl, an alkyl silyl, a heterocycle, or a substituted heterocycle;
R₅ is H, OH, a —OC(O)Rₓ, group, a —C(O)Rₓ, or a —C(O)ORₓ, group, where Rₓ is a C₂ to C₁₅ alkyl, preferably a C₂ to C₈ alkyl;
R₆ is H, O (carbonyl group), carboxyl (carboxylate group), a —OC(O)Rₓ group, a —C(O)Rₓ, or a —C(O)ORₓ, group, where Rₓ, is defined above;
X is H or is OR_{b}, where R_{b} is either H, an alkyl, a substituted alkyl, an aryl, a substituted aryl, a heterocycle, or a substituted heterocycle.

Preferably, R₁, R₂, R₃, R₄ are Me, R₅ is H or OH, more preferably OH, R₆ is H, R₇ is OH, and X is OH. Preferably, in certain embodiments, X is H, OH, O(C₁-C₄) alkyl, O-benzyl, O-trialkylsilyl (e.g., C₁-C₄ alkyl, such as methyl, ethyl, i-propyl or t-butyl, especially trimethyl silyl, tri-iPr silyl, dimethyl t-butyl silyl, O-diarylalkylsilyl (such as diphenyl t-butyl, among others) or O-triarylsilyl.

Preferred compounds of the invention include synthetic tyloindicine analogues of formulae (III), (IV), (V), and (VI) illustrated in FIG. 15, designated, respectively, as NSC-717334, NSC 712822, NSC 717336, and DCB-3501 and DCB-3503 (DCB-3503 is also referred to synonymously as NSC 716802 or ZH-152). The compounds of formula (VI) (DCB 3501 and DCB 3503) are particularly preferred, with DCB 3503 (hydroxyl “down”) being particularly preferred. Additional preferred compounds are those set forth in Figure a, Table 1. Compounds DCB 3501 and 3503 have exhibited extraordinary antitumor activity, e.g., when used in the National Cancer Institute (NCI) screen. In particular, compounds according to the present invention exhibit significant anti-tumor activity against a variety of drug-resistant tumors/cancer and in particular, multiple drug resistant tumors.

The invention also provides anti-neoplastic (including anti-cancer), anti-inflammatory and anti-viral (anti-EBV) pharmaceutical compositions comprising these novel tyloindicine analogues, methods of using these pharmaceutical compositions to treat a wide variety of neoplastic, and inflammatory conditions as well as EBV infections and EBV-related lymphoma and cancer, and processes for making tyloindicines, including the novel tyloindicine analogues of the instant invention.

When used in accordance with the instant invention in the National Cancer Institute (“NCI”) human cell-line tumor panel, tylo F and tylo G ranked, respectively, as the most potent anticancer agents examined in a screen that includes some 33,744 compounds and data from fifty-four cell lines of the DTP (Developmental Therapeutics Program) of the NCI. The ranking system was based on the average concentration required to yield a total growth inhibition (TGI) in the numbers of cell lines of the screen (54 for tylo F and tylo G). The concentrations required to reach 50% growth inhibition (GI₅₀) are <10⁻¹⁰ M for both compounds, a value that is at least two orders of magnitude lower than the next-nearest competitor. In fact, for many cell lines, the data were off scale; designated as <10⁻¹⁰ M.

When LC₅₀ values (the concentration which decreases 50% of the initial cells seeded) of both tylo F and G were used in accordance with the instant invention against tumor cells of the DTP panel, it was determined that the values for several of the melanoma cell lines and the lung (small and non-small) cancer cell lines are two orders of magnitude less than those for the other cell lines, evidencing the selectivity of these two compounds when used in accordance with the invention against some melanomas and/or lung cancers.

Additionally, the antitumor screen COMPARE was used to test anti-cancer activity in vitro. COMPARE is described in Paull, K D.; Hamel, Cancer Chemotherapeutic Agents; Foye, W. O., Ed.; American Chemical Society: Washington, 1994, p 9-45 (Chapter 2). COMPARE is a program (a pattern-recognition algorithm) that ranks the anticancer activity of compositions with those of the entire NCI database; it was used to determine the activity of tylo F and tylo G when applied in accordance with the instant invention. Tylo F and Tylo G exhibited patterns of activity unlike those of standard antitumor compounds, i.e., were “COMPARE negative”, and proved to be distinctly different in their (a) chemical structures and (b) mechanism of action from all known antitumor compounds (e.g., alkylators, DNA-interactive compounds, and topoisomerase-active agents).

Without any intention to limit the invention by any theory, given the potency of the tylos and tylo analogues of the instant invention, and the potency of the epimers, pharmaceutically acceptable salts, solvates, or polymorphs thereof, when used in accordance with the instant invention to inhibit cell growth of a variety of cell lines with GI₅₀ levels of less than 10⁻¹⁰ M, it may be that these compounds interact tightly with one or more proteins which play an important role in cell growth. It is possible that this interaction triggers a downstream event causing cell arrest. In the cell lines, such as melanoma or lung cancer, which are killed by these two compounds in accordance with the invention, the downstream event triggered through the interaction of compounds with their putative target protein(s) may prove to be different from that of cells that are only growth arrested.

Alternatively, it is possible that the mechanism responsible for cell death attributable to application of the instant invention could be different from that for cell growth arrest. In such a case, the existence of more than one binding protein is possible. The binding protein that has the highest binding affinity may be responsible for cell arrest and is shared in all cancer cells. The lower affinity binding protein may be responsible for cell death and may be present only in those sensitive (with respect to cell death) melanoma or lung tumor cell lines. Again, such theoretical postulates in no way limit the full scope of the instant invention as disclosed and claimed herein.

In another aspect, the invention includes the use of tylos and tylo analogues of the instant invention, and pharmaceutically acceptable salts, solvates, or polymorphs thereof, in vivo as “warheads” for antibodies or proteins targeted on tumor cells. Appropriate ligands for such utilities may readily be determined in connection with affinity chromatographic isolation of proteins and as protein-dug conjugate prodrugs.

Embodiments of the instant invention include the use of tylos and tylo anologues of the instant invention, and pharmaceutically acceptable salts, solvates, or polymorphs thereof, in the treatment of a wide variety of tumor cells, wherein the mechanism of action of the tyloindicines may be different when applied against different tumors. Activities of tyloindicines when used in accordance with the instant invention will not be influenced by MDR (gp170) and MRP (multiple drug resistance protein) overexpression. Tylos and tylo epimers of the instant invention, and pharmaceutically acceptable salts, solvates, or polymorphs thereof, also prove active against cancer cells that exhibit resistance to other drugs, such as hydroxyurea, gemcitabine, Topo-I drugs as well as Topo-II drugs.

Further, in another aspect of the invention, the applicants have determined that tylos and tylo anologues of the instant invention, and pharmaceutically acceptable salts, solvates, or polymorphs thereof, exhibit a potent activity against NF-κK mediated transcription and therefore have a related utility in the treatment of inflammation, autoimmune disorders and diseases or symptoms associated with activation of NF-κB, such as arthritis, asthma, fibrosis, and nephritis.

Further, in another aspect of the invention, the applicants have determined that tylos and tylo anologues of the instant invention, and pharmaceutically acceptable salts, solvates, or polymorphs thereof, can be used in combination with other anti-cancer agents, or other chemicals for treatment of inflammation related diseases.

Further, in another aspect of the invention, the applicants have determined that tylos and tylo anologues of the instant invention, and pharmaceutically acceptable salts, solvates, or polymorphs thereof, can be used in prodrugs that improve the solubility, stability, as well as absorption and pharmacokinetic profile.

The present invention may also be used prophylatically to either prevent or reduce the likelihood of the occurrence of an EBV infection or an EBV-related lymphoma or cancer in a patient.

### BRIEF DESCRIPTION OF THE DRAWINGS

FIG. 1 illustrates the following referred to in the Examples herein:
“A. Chemical structure” provides the structural formulae for (+)-(S)-tylophorine (also referred to herein as compound “III-2” and DCB-3500); DCB-3501; “compound II-3a of FIG. 15”; and, in FIGS. 18-22, as “ZH-152”); DCB-3502 (also referred to herein as “compound II-2); and DCB-3503 (also referred to herein as “NSC 716802”; “compound II-3b” of FIG. 15; and “ZH-152”).
“Table 1, B and C” provides EC₅₀ and LD50 values for use of DCB-3500, DCB-3501, DCB-3502, and DCB-3503 against KB and HepG2 cancer cell lines resistant to various anticancer drugs, including VP-16 (etoposide), VCR (vincristine), CPT (camptothecin), and DOX (doxorubicin).
“Table 2 A, and B, and Table 3” illustrate that KB and HepG2 cancer cell lines are inhibited by the compounds of the instant invention. Table 2 shows the effect of EC₅₀ of DCB-3500, 3501, 3502 and 3503 on the growth inhibition of KB cells and its drug resistant cells. The results indicate that DCB-3500, 3501, 3502, and 3503 have no cross-resistance with conventional anticancer drugs as indicated in the table, implying that this class of compound may have a adopt a novel mechanism for anti-cancer, and may target a novel protein. Table 3 shows the impact of DCB-3500 and 3503 on cell cycle progression KB and HepG2 cells were treated with several concentrations of DCB-3500 and DCB-3503 for 24 h. At the end of treatment, cells were washed, resuspended in PBS and stained with propidium iodide containing Rnase A for flow cytometric analysis. Data were analyzed using Modfit software. The results set forth in table 3 show that DCB-3500 and 3503 treatment could induce S phase accumulation in KB cells, but not in HepG2 cells.

“FIG. 1(a)” in graphs A and B illustrates the effect of DCB-3500 and DCB-3503 on the growth of HepG2 tumor xenografts in nude mice. The following legend applies to FIG. 1(a): (A) Effect of DCB-3503 on the growth of HepG2 tumor in nude mice, (▪) control, (▴) DCB-3500 and (▾) DCB-3503. (B) Effect of DCB-3500 and DCB3503 on the body weight of nude mice. HepG2 cells (2×10⁶) were implanted subcutaneously into nude mice (average body weight is 20 g) for 10 days. Treatment was carried out by using I.P. to inject 3 dosages of DCB-3500 and DCB-3503 at 30 mg/kg in every 8 hours on day 11 after tumor implanted. Tumor weight was estimated by using the equation: Length of tumor x (wide of tumor/2)².

FIG. 2 illustrates confocal micrographs of the effect of various anticancer drugs and DCB-3500 and DCB-3503 on KB and HepG2 cells as described in the Examples herein. This figure shows the regulation of p53 in response to conventional chemotherapeutic drugs and 3500, 3503. The cells were treated with conventional anti-cancer drugs and DCB-3500 and 3503 as indicated for 24 h, p53 expression level were analyzed by confocal microscope using an anti-p53 antibody.

FIG. 3 illustrates flow cytometric data on the effect of DCB-3503 on KB and HepG2 cells as described in the Examples herein. As presented, 2×10⁶ untreated or 3503 treated KB and HepG2 cells were stained with Alexa Fluor 488 annexin V and propidium iodide and were analyzed by flow cytometer. Apoptotic cells (lower right panel) showed green fluorescence. Necrotic cells (upper right panel) showed both red and green fluorescence.

FIG. 4 illustrates the growth inhibitory effect of (+)-(S)-tylophorine (“III-2” or DCB-3500) and analogues DCB-3501, DCB-3502 and DCB-3503 on HepG2 cells as described in the Examples herein. A) Growth inhibitory effect of 3500 and its analogs in HepG2 cells. HepG2 cells were treated with DCB-3500, 3501, 3502, and 3503 as indicated for 24 h, then drugs were taken away, and cell growth was monitored. (B) HepG2 cells were treated with or without DCB-3500 for 24 h, drug was taken away, and AFP expression was monitored by confocal microscopic analysis using an anti-AFP antibody. (C) HepG2 cells were treated without or with DCB-3500 as indicated for 24 h, drug was taken away, and after 5 days albumin expression was detected by confocal microscope using an anti-albumin antibody.

FIG. 5A-G illustrate the potent activity of (+)-(“S”)-tylophorine (“III-2” or DCB-3500), DCB-3502 and DCB-3503 against NF-κB mediated transcription as determined in a firefly luciferase assay as described in the Examples herein. HepG2 cells were transiently transfected with firefly luciferase reporter vectors pMyc-TA-luc, pE2F-TAO-luc, pAP1-luc, pCRE-luc, or pBIIX-luc (containing two tandemly repeated NF-kB binding sites), respectively, along with internal control vector phRL-luc which is a promoterless renilla luciferase reporter vector. The day after transfection, cells were pretreated with increasing concentrations of 3500 for 1 h, then cells were stimulated with serum for 24 h, or TPA, forskolin or TNFa for 6 h Firefly and renilla uciferase activities were measured using Promega's dual-luciferase assay kit Data presented is firefly luciferase activity.

FIG. 6, in Scheme I, illustrates the synthesis of compounds of the instant invention.

FIG. 7, in Scheme II, illustrates the synthesis of compounds of the instant invention.

FIG. 8, in Scheme III, illustrates a confirmation of the utility of Schemes 1 and 2 illustrated in FIGS. 6 and 7.

FIG. 9, in Scheme IV, illustrates Synthesis of tyloindicine G in accordance with the instant invention.

FIG. 10, in Scheme V, illustrates alternative hydroxylation schemes using a Polonovsky reaction in accordance with the instant invention.

FIG. 11, in Scheme VI, illustrates synthesis of tyloindicine F in accordance with the instant invention.

FIG. 12, in Scheme VII, illustrates synthesis of tyloindicine I in accordance with the instant invention.

FIG. 13, in Scheme VIII, illustrates synthesis of tyloindicine H in accordance with the instant invention.

FIG. 14, in Scheme IX, illustrates synthesis of congeners in the tyloindicine series in accordance with the instant invention.

FIG. 15 illustrates the structural formulae of tyloindicine analogues NSC 717334, NSC712822, NSC 717336, and NSC 716802 (DCB-3501 and DCB-3503) of the instant invention.

FIG. 16, in Scheme X, illustrates synthesis of tyloindicine G in accordance with the instant invention.

FIG. 17, in Scheme XI, illustrates synthesis of an activated CH-Sepharose-NSC-717335 prodrug.

FIG. 18 describes cross resistance studies in KB cell lines using DCB-3503. In conclusion: Cells which become resistent to VP-16, VCR, CPT or DOX are still sensitive to ZH-152.

FIG. 19 illustrates the effect of DCB-3503 (ZH-152) in clonogenic assays. As depicted, cells were seeded at 5×10⁴ per well, then DCB-3503 was added at concentrations ⅓, 1× and 3× the IG₅₀. After a 24 h treatment, the cells were recounted and seeded into a fresh 6 well plate at 200 cells per well. After 8 generations of time the colonies were stained with methylene blue and counted. The cloning efficiency for HepG2 was 10% and for KB was 94%. Both cell lines were exposed to DCB-3503 with the concentration indicated for 24 h. The loss of clonegenic efficiency of cells post drug treatment is shown. HepG2 is much more sensitive than KB cells. This supports the previous observation using a different procedure.

FIG. 20 illustrates the effect of DCB-3503 on KB and HepG2 cell growth. DCB-3503 slows down the cell progress in S-phase of both cell lines. Thus, the growth inhibition of these two cell lines by DCB-3503 is due to the inhibition at targets responsible for S-phase progression. Additional biochemical determinants may play a role in the preferential killing (loss of clonogenecity) of HepG2 to the of KB.

FIG. 21 illustrates toxicity studies of DCB-3503 in C57BL/6 mice. DCB-3503 shows a toxicity in this study of 10 mg/kg by causing weight loss.

FIG. 22 illustrates toxicity and tumor growth inhibition studies using DCB-3503. DCB-3503 shows potent inhibitory activity against HepG2 growth in nude mice (single experiment).

### DETAILED DESCRIPTION OF THE INVENTION

As used herein, the following terms have the following respective meanings.

The term “alkyl” is used herein to refer to a fully saturated monovalent hydrocarbon radical containing carbon and hydrogen, and which may be a straight chain, branched or cyclic. Examples of alkyl groups are methyl, ethyl, n-butyl, n-heptyl, isopropyl, 2-methylpropyl, cyclopropyl, cyclopropylmethyl, cyclobutyl, cyclopentyl, cyclopentylethyl and cyclohexyl. “Cycloalkyl” groups refer to cyclic alkyl groups such as cyclopropyl, cyclobutyl, cyclopentyl and cyclohexyl. C₁-C₆ alkyl groups are preferably used in the present invention; C₁ to C₃ are particularly preferred.

The term “substituted alkyl” refers to alkyl as just described which include one or more functional groups such an alkyl containing from 1 to 6 carbon atoms, preferably a lower alkyl containing 1-3 carbon atoms, aryl, substituted aryl, acyl, halogen (i.e., alkyl halos, e.g., CF₃), hydroxy, alkoxy, alkoxyalkyl, amino, alkyl and dialkyl amino, acylamino, acyloxy, aryloxy, aryloxyalkyl, carboxyalkyl, carboxamido, thio, thioethers, both saturated and unsaturated cyclic hydrocarbons, heterocycles and the like. The term “substituted cycloalkyl” has essentially the same definition as and is subsumed under the term “substituted alkyl” for purposes of describing the present invention.

The term “aryl” refers to a substituted or unsubstituted monovalent aromatic radical having a single ring (e.g., phenyl) or multiple condensed rings (e.g., naphthyl). Other examples include heterocyclic aromatic ring groups having one or more nitrogen, oxygen, or sulfur atoms in the ring, such as imidazolyl, furyl, pyrrolyl, pyridyl, thienyl and indolyl, among others. The term “heteroaryl” is subsumed under the more general term “aryl”.

The term “substituted aryl” refers to an aryl as just described that contains one or more functional groups such as lower alkyl, acyl, aryl, halogen, alkylhalos (e.g., CF₃), hydroxy, alkoxy, alkoxyalkyl, amino, alkyl and dialkyl amino, acylamino, acyloxy, aryloxy, aryloxyalkyl, carboxyalkyl, carboxamido, thio, thioethers, both saturated and unsaturated cyclic hydrocarbons, heterocycles and the like.

“Heterocycle” or “heterocyclic” refers to a carbocylic ring wherein one or more carbon atoms have been replaced with one or more heteroatoms such as nitrogen, oxygen or sulfur. Examples of heterocycles include, but are not limited to, piperidine, pyrrolidine, morpholine, thiomorpholine, piperazine, tetrahydrofuran, tetrahydropyran, 2-pyrrolidinone, δ-velerolactam, δ-velerolactone and 2-ketopiperazine, among numerous others.

The term “substituted heterocycle” refers to a heterocycle as just described that contains one or more functional groups such as C₁-C₄ alkyl, acyl, aryl, cyano, halogen, hydroxy, alkoxy, alkoxyalkyl, amino, alkyl and dialkyl amino, acylamino, acyloxy, aryloxy, aryloxyalkyl, carboxyalkyl, carboxamido, thio, thioethers, both saturated and unsaturated cyclic hydrocarbons, heterocycles and the like. In other instances where the term “substituted” is used, the substituents which fall under this definition may be readily gleaned from the other definitions of substituents which are presented in the specification as well the circumstances under which such substituents occur in a given chemical compound.

The term “epimer” is used herein to designate a compound that differs in confugation at only one of two or more asymmetric centers.

The term “one or more substituents” as used herein refers to a number of substituents that equals from one to the maximum number of substituents possible based on the number of available bonding sites.

The term “enantiomers” refers to two stereoisomers of a compound which are non-superimposable mirror images of one another. “Stereoisomers” refers to compounds which have identical chemical constitution, but differ with regard to the arrangement of their atoms or groups in space. An “enantioselective process” is one which favors production of one of the two possible enantiomers of a reaction product. “Enantiopure” or “enantomerically pure” means a pure stereoisomer uncontaminated by its enatiomer. A “racemic” mixture is a mixture of two enantiomers.

The term “halogen group” as used herein means F, Cl, Br or I.

The term “patient” is used throughout the specification to describe an animal, preferably a human, to whom treatment, including prophylactic treatment, with the compositions according to the present invention is provided. For treatment of those infections, conditions or disease states which are specific for a specific animal such as a human patient, the term patient refers to that specific animal.

The term “neoplasia” is used to describe the pathological process that results in the formation and growth of a neoplasm, i.e., an abnormal tissue that grows by cellular proliferation more rapidly than normal tissue and continues to grow after the stimuli that initated the new growth cease. Neoplasia exhibits partial or complete lack of structural organization and functional coordination with the normal tissue, and usually forms a distinct mass of tissue which may be benign (benign tumor) or malignant (carcinoma). The term “cancer” is used as a general term to describe any of various types of malignant neoplasms, most of which invade surrounding tissues, may metastasize to several sites and are likely to recur after attempted removal and to cause death of the patient unless adequately treated. As used herein, the term cancer is subsumed under the term neoplasia. The term “drug resistant cancer” or “multiple drug resistant cancer” is used throughout the specification to describe cancers which are resistant to one or more traditional cancer drugs, for example, hydroxyurea, gemcitabine, Topo-I drugs as well as Topo-II drugs, among numerous others. Compounds according to the present invention may be administered in the presence (coadministered) or absence of these agents.

The terms “inflammatory disorder” or “autoimmune disorder” as used herein include disorders associated with NF-κB mediated transcription, transplantation rejection (e.g., renal allograft rejection, a cardiac allograft rejection, and transplantation-associated vasculopathy), nephritis (e.g., acute glomerulonephritis, lupus nephritis and tubulointerstitial nephritis), asthma (e.g., allergic asthma), respiratory distress syndrome, gastritis (e.g., indomethacin-induced gastritis), rheumatoid diseases (e.g., arthritis or lupus), autoimmune diseases (e.g., vasculitis, diabetes, and HIV/AIDS), sepsis, thrombosis, and coronary artery disease (e.g., restenosis after angioplasty or by-pass surgery and ischemia). In particular, the compounds of the instant invention are useful in treating disorders associated with the activation of NF-κB, including rheumatoid arthritis, inflammatory bowel disease, asthma, dermatitis including psoriasis and atopic dermatitis, autoimmune diseases, tissue and organ rejection, Alzheimers disease, Hodgkin's disease, viral infections including AIDS, and Ataxia Telangiestasia.

The term “pharmaceutically acceptable salt” is used throughout the specification to describe a salt form of one or more of the compositions (and in particularly preferred aspects according to the present invention, phosphate salts) herein which are presented to increase the solubility of the compound in saline for parenteral delivery or in the gastric juices of the patient's gastrointestinal tract in order to promote dissolution and the bioavailability of the compounds. Pharmaceutically acceptable salts include those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the pharmaceutical art. Sodium and potassium salts are particularly preferred as neutralization salts of carboxylic acids and free acid phosphate containing compositions according to the present invention. The term “salt” shall mean any salt consistent with the use of the compounds according to the present invention. In the case where the compounds are used in pharmaceutical indications, including the treatment of neoplasia, including cancer, the term “salt” shall mean a pharmaceutically acceptable salt, consistent with the use of the compounds as pharmaceutical agents.

The term “inhibitory effective concentration” or “inhibitory effective amount” is used throughout the specification to describe concentrations or amounts of compounds according to the present invention which substantially or significantly inhibit the growth or replication of susceptible neoplasias.

The terms “therapeutic effective amount”, or “therapeutically effective amount” shall mean an amount or concentration of a compound according to the present invention which is effective within the context of its administration or use, including, for example, the treatment of neoplasias, inflammatory disorders or autoimmune disorders. Thus, the term “effective amount” is used throughout the specification to describe concentrations or amounts of compounds according to the present invention which may be used in context to produce a favorable result within the context of the compound's use, including, for example a change in the disease or condition treated, whether that change is a remission, a decrease in growth or size of cancer or a tumor or a favorable physiological result, or the like, depending upon the disease or condition treated.

The term “preventing effective amount” is used throughout the specification to describe concentrations or amounts of compounds according to the present invention which are prophylactically effective in preventing, or reducing the likelihood of an autoimmune disorder including inflammatory disorders or an EBV infection or a related condition or disease state.

The term “effective amount” is used throughout the specification to describe amounts of compounds or compositions used or administered within context to effect an intended result This term subsumes other terms which describe effective amounts which are used in different contexts.

The term “Epstein Barr virus” or (EBV) is used throughout the specification to describe the virus found in cell cultures of Burkitt's lymphoma. Structurally, EBV is similar to that of other herpes viruses- it has a double-stranded DNA genome contained within a nucleocapsid, which is surrounded by a lipid envelope containing viral glycoproteins. A tegument protein occupies the space between the envelope and the nucleocapsid. EBV is the causative agent in infectious mononucleosis. Epstein-Barr virus is also recognized as a causative agent of B-cell proliferative diseases, lymphoproliferative syndrome, nonfamilial monophagocytic syndrome and is linked to a variety of disease states, including a rare progressive mononucleosis-like syndrome and oral hair leukoplakia in AIDS patients. EBV has also been associated with certain types of cancer such as Burkitt's lymphoma, nasopharyngeal carcinoma, Hodgkin's disease, EBV-associated T-cell lymphoma and nasal T-cell lymphoma. Certain patients, in particular, those with suppressed immune systems such as AIDS patients and organ transplant patients who are being treated with immunosuppressive agents, are particularly susceptible to EBV manifestations, especially the development of EBV-associated lymphomas.

The term “coadministration” or “combination therapy” is used to describe a therapy in which at least two active compounds in effective amounts are used to treat a a tumor and/or cancer, or an autoimmune disorder, condition or disease state. Although the term coadministration preferably includes the administration of two active compounds to the patient at the same time, it is not necessary that the compounds be administered to the patient at the same time, although effective amounts of the individual compounds will be present in the patient at the same time.

Compounds according to the present invention may be used in pharmaceutical compositions having biological/pharmacological activity for the treatment of, for example, neoplasia, including cancer, as well as a number of other conditions and/or disease states, as intermediates in the synthesis of compounds exhibiting biological activity as well as standards for determining the biological activity of the present compounds as well as other biologically active compounds. These compositions comprise an effective amount of any one or more of the compounds disclosed hereinabove to be used within the context of administration, optionally in combination with a pharmaceutically acceptable additive, carrier or excipient.

A further aspect of the present invention relates to the treatment of neoplasia, including cancer (and in particular drug resistant or multiple drug resistant cancer), comprising administering to a patient in need thereof an effective amount of a compound as described hereinabove, optionally in combination with a pharmaceutically acceptable additive, carrier or excipient. The present invention also relates to methods for inhibiting the growth of neoplasia, including a malignant tumor or cancer comprising exposing the neoplasia to an inhibitory or therapeutically effective amount or concentration of at least one of the disclosed compounds. This method may be used therapeutically, in the treatment of neoplasia, including cancer or in comparison tests such as assays for determining the activities of related analogues as well as for determining the susceptibility of a patient's cancer to one or more of the compounds according to the present invention. Primary utility resides in the treatment of neoplasia, including cancer, especially including lung cancer, breast cancer and prostate cancer, among others.

A preferred therapeutic aspect according to the present invention relates to methods for treating neoplasia, including benign and malignant tumors and cancer in animal or human patients, and in preferred embodiments, cancers which have developed drug resistance, including, for example, multiple drug resistant breast cancer comprising administering therapeutically effective amounts or concentrations of one or more of the compounds according to the present invention to inhibit the growth or spread of or to actually shrink the neoplasia in the animal or human patient being treated.

Cancers which may be treated using compositions according to the present invention include, for example, stomach, colon, rectal, liver, pancreatic, lung, breast, cervix uteri, corpus uteri, ovary, prostate, testis, bladder, renal, brain/cns, head and neck, throat, Hodgkins disease, non-Hodgkins leukemia, multiple myeloma leukemias, skin melanoma, acute lymphocytic leukemia, acute mylogenous leukemia, Ewings Sarcoma, small cell lung cancer, choriocarcinoma, rhabdomyosarcoma, Wilms Tumor, neuroblastoma, hairy cell leukemia, mouth/pharynx, oesophagus, larynx, melanoma, kidney and lymphoma, among others. Compounds according to the present invention are particularly useful in the treatment of lung cancer, breast cancer and prostate cancer and drug resistant forms of cancer, in particular multiple drug resistant forms.

In the present methods, in certain preferred embodiments, it has been found advantageous to coadminister at least one additional anti-neoplastia agent for the treatment of neoplasia, including cancer. In these aspects according to the present invention, an effective amount of one or more of the compounds according to the present invention is co-administered along with an effective amount of at least one additional anti-neoplastia/anti-cancer agent such as, for example traditional and non-traditional anti-tumor or anti-cancer agents for example, etoposide (VP-16), cis-platin (cisDDP), carboplatin, lobaplatin, ormaplatin, oxaplatin, hexamethylmalamine, NLCQ-1, mephalan (L-PAM), dihydroxybusulfan and other alkylating agents, such as cyclophosphamide (CPM), among others, daunorubicin, doxorubicin, mitomycin, adriamycin, camptothecin, vinca alkaloids (vincristine and vinblastine), hydroxyurea, gemcitabine, Topo-I and Topo II drugs, polynucleotides and oligonucleotides (sense and anti-sense), taxol and other taxoid anti-tumor agents as disclosed in, for example, U.S. Pat. No. 6,500,858, relevant portions of which are incorporated by reference hereof, methacycline compounds, such as those disclosed in U.S. Pat. No. 6,500,812, relevant portions of which are incorporated by reference hereof, anti-angiogenesis agents, azaindole derivatives as described in U.S. Pat. No. 6,486,322, other compositions as described in U.S. Pat. No. 6,488,9312, dibenzofluorene derivatives as described in U.S. Pat. No. 6,479,662, relevant portions of all of said patents being incorporated by reference hereof, temozolomide, AP/AMP and their prodrug forms, among numerous others to a patient for the treatment of a tumor and/or cancer.

The compositions of the present invention may be formulated in a conventional manner using one or more pharmaceutically acceptable carriers. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally, or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of novel tylo of the instant invention that may be combined with the carrier materials to produce a single dosage form will vary depending upon the host treated, the particular mode of administration. Preferably, the compositions should be formulated so that a dosage of between about 0.5 and 200 mg/kg bodoy weight/day, more preferably about 1 to about 100 mg/kg body weight/day of the novel tylo can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

Chemistry

The novel compounds of the instant invention were generally prepared in the following manner.

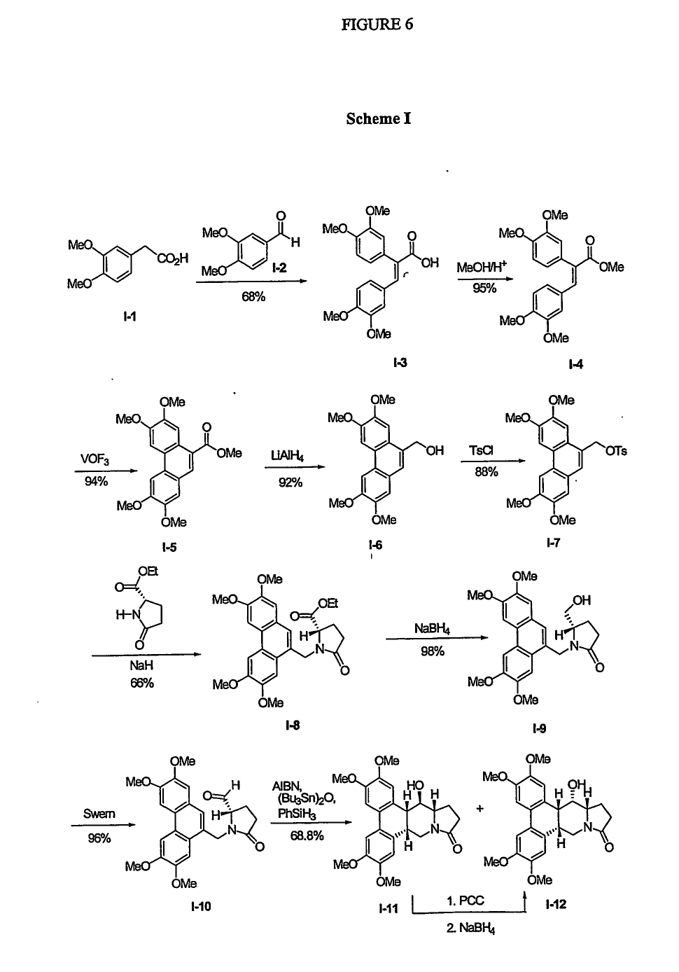

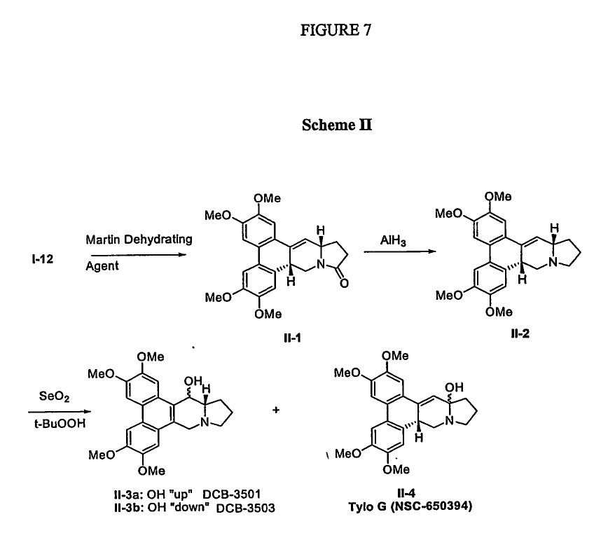

Schemes I and II, depicted in FIGS. 6 and 7, illustrate synthesis of the tylo G skeleton. As illustrated in Scheme I, FIG. 6, in this synthesis, the sensitive 12a-OH group was installed late in the sequence. Condensation of I-1 with I-2 (Et₃N—Ac₂O) gave the known α,β-unsaturated carboxylic acid I-3, Ihara, M.; et al., Stereocontrolled Synthesis of Quinolizidines and Indolizidines Using Trialkylsilyl Triflurormethanesulphonate: Total Synthesis of -Tylophorine. J. Chem. Soc., Chem. Commun. 1985, 1159-1160, which was then converted to its methyl ester I-4. VOF₃ ring closure then afforded I-5 in high yield. LiAlH₄ reduction, then tosylation of the resulting alcohol I-6 gave I-7, which was in turn displaced with ethyl (+)-(S)-2-pyrrolidine-5-carboxylate sodium salt (Aldrich or synthesized) to give optically active I-8. Reduction of I-8 with NaBH₄ generated the alcohol I-9, which was oxidized (Swem) to the aldehyde I-10 in high yield. Free-radical mediated reductive cyclization, Hays, D. S. et al., Organotin hydride catalyzed carbon-carbon bond formation: Radical-mediated reductive cyclization of enals and enones. J. Org. Chem. 1996, 61, 4-5; Hays, D. S.; et al., The development of a new catalytic process: Bu₃SnH-catalyzed reductive cyclization of enals and enones. Tetrahedron 1999, 55, 8815-8832, of I-10 then gave an epimeric mixture (1.2:1) of alcohols I-11 and I-12 that were separated by silica gel chromatography.

An X-ray crystal structure was obtained for I-11 that verified the structure as that shown. This established the stereochemistry at the benzylic position, linking the stereochemistry with that of the natural tyloindicines that are typified by large negative optical rotations {[α]_{D}²²−104° for I-11}.

In FIG. 6, Scheme I, Compound I-11 was converted to its epimer I-12 by Swem oxidation: NaBH₄ reduction.

Referring to Scheme II, FIG. 7, Martin sulfurane dehydration, Arhart, R. J.; Martin, J. C. Sulfuranes. V. Chemistry of sulfur (IV) compounds. Dialkoxydiarylsulfuranes. J. Am. Chem. Soc. 1972, 94, 4997-5003, then gave the alkene II-1, which upon reduction with AlH₃ gave the alkene II-2. Attempts to install the 12a-OH group of tylo G via SeO₂ hydroxylation led to isolation of the benzylic alcohols II-3a and II-3b whose structure were confirmed by MS and NMR spectroscopy. NMR showed that the correct 12a-OH compound (tylo G) is also being formed, but is perhaps undergoing decomposition under the reaction/isolation conditions used in the SeO₂ reaction. Alcohols II-3a and H-3b may also be synthesized by the procedure of Buckley and Rapoport, Buckley, T. F.; Rapoport, H. α-Amino acids as chiral educts for asymmetric products. Chirally specific syntheses of tylophorine and cryptopleurine. J. Org. Chem. 1983, 48, 42224232.

As a further demonstration of the utility of the synthetic route shown in Schemes I and II, as well as to demonstrate (before X-ray studies were carried out on I-11) that it is indeed the correct enantiomers that are being worked with, Scheme III illustrated in FIG. 8 was performed from I-11 to (+)-(S)-tylophorine, a compound that has been synthesized and has reported antitumor (breast) activity, although nothing as potent as the tyloindicines.

### Synthesis of Tyloindicines F, G, H, and I

(a) General Considerations. Synthetic schemes for each of tyloindicines F, G, H, and I are described herein. Preliminary studies have resulted in a firm elucidation (single-crystal X-ray diffraction analysis) of the stereochemistry for a system that matches that reported in the literature for these compounds. The fact that synthetic analogues of the instant invention NSC-716802 and NSC-717335 have potent antitumor activity demonstrates that the synthetic schemes shown herein provide the correct stereochemistry. Tylos F and G are subject to facile epimerization. Tertiary OH groups are indicated herein with wavy bonds in the schemes to indicate the thermodynamic mixture of epimers. It is possible, in light of the fact that the activity demonstrated by the intermediate NSC-717335 (compound II-2, Scheme II), together with the activities observed for tylos H and L that the tertiary OH functions may not have a significant influence on antitumor activity. The role of these OH groups can be established via side-by-side antitumor testing with nonhydroxylated counterparts.

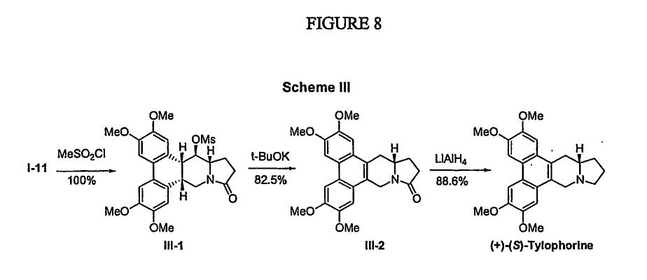

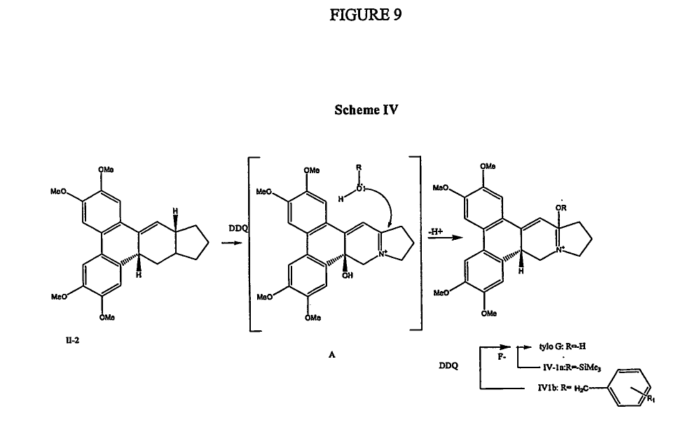

(b) Synthesis of tyloindicine G The synthetic scheme for tyloindicine G is based upon selectively generating an iminium ion that provides a suitable species for nucleophilic attack at C-12a by an oxygen-containing reagent. To this end, a process was developed that shows allylic (12aH) selectivity over the allylic-benzylic H and generates iminium ion A by DDQ oxidative abstraction of H-12a on II-2, as shown in FIG. 9, Scheme IV. Addition of MeOH then gives the 12-OMe compound that by 1D and 2D NMR spectroscopy and MS is indicated to be the expected stereochemistry and structure as shown. H₂O may also be used as the reagent to directly generate the 12a-OH compound. Alternatively Gassman-dry-OH generated by H₂O and t-BuOK in THF is a possibility. An alternative approach is to use Me₃SiOH, which allows F deprotection, or one of the benzylic alcohols (e.g., R¹=2,6-dimethoxybenzyl-, 4-methoxybenzyl-, or 2-naphthylmethyl ethers) that are removable with either DDQ or CAN under neutral conditions. It is recognized that the tylo G structure has a benzylic function and could react with DDQ. However, given the fact that (1) it is possible to generate the iminium species selectively with DDQ and that (2) some of the aforementioned substituted benzyl ethers are exceptionally labile to DDQ, the reaction scheme described above is supportable. Preferably, the reaction is carefully conducted by (1) generating the iminium ion with little or no excess of DDQ, and (2) adding the alcohol at 78° C. Allyl alcohol, whose resulting allyl ether can be removed with an iridium catalyst isomerization-mild hydrolysis of the 2-propenyl ether, is another alternative. There is considerable precedent that such alcohols and their methyl ethers enjoy stability and can be isolated.

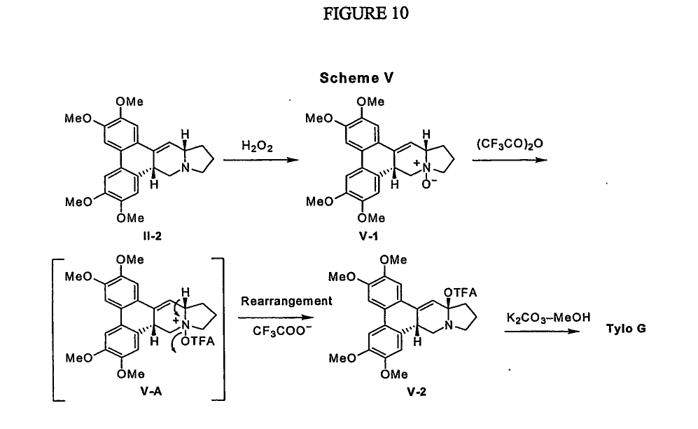

Alternative hydroxylation schemes are also within the scope of the invention. These include using a Polonovsky reaction, Grierson, D. The Polonovsky Reaction. Org. React. 1990, 39, 85-295, which is carried out on II-2 as shown in FIG. 10, Scheme V. Thus II-2 is converted to the N-oxide V-1 and then trifluoroacetylated to give the N-OTFA intermediate V-A, which rearranges to give the O-TFA derivative V-2. Deprotection under K₂CO₃-MeOH treatment then furnishes tylo G. Full characterization of the products may be made via MS and NMR spectroscopy, including a determination of the orientation and/or interconversion of the 12a-OR and 12a-OH groups.

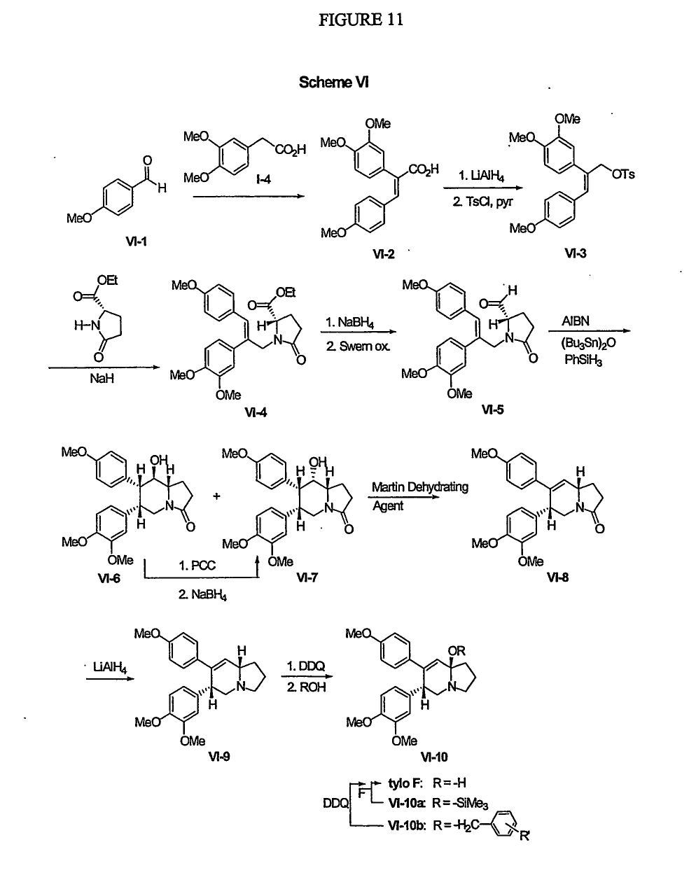

(c) Synthesis of tyloindicine F The methodology developed for tylo G is likewise applied to the tylo F synthesis, as shown in FIG. 11, Scheme VI. Thus condensation of 4-methoxybenzaldehyde (VI-1) with 3,4-dimethoxyphenylacetic acid (14) gives the carboxylic acid VI-2, which upon LiAlH₄ reduction and tosylation of the resulting alcohol, gives the tosylate VI-3. Displacement of the tosylate with ethyl (+)-(S)-2-pyrrolidine-5-carboxylate sodium salt gives adduct VI-1. Reduction of the ester function, followed by Swem oxidation, then gives the aldehyde VI-5. The aldehyde is reductively cyclized to give the saturated alcohols VI-6 and VI-7. As in the tylo G synthesis, VI-6 can be converted to VI-7 via the sequence of PCC oxidation-NaBH₄ reduction. As with the tylo G examples, X-ray analysis confirms the stereochemistry.

Martin sulfurane dehydration carried out on VI-7 then gives the unsaturated intermediate VI-8, which is reduced with LiAlH₄ to give VI-9, the tylo F analogue of the antitumor active NSC-717335. Installation of the HO- or PO-functions at the indolizidine C-8a is then accomplished via the procedures outlined for tylo G, above. Full characterization of the products, including orientation of the 8a-OR and 8a-OH groups, may be made by MS and NMR spectroscopy.

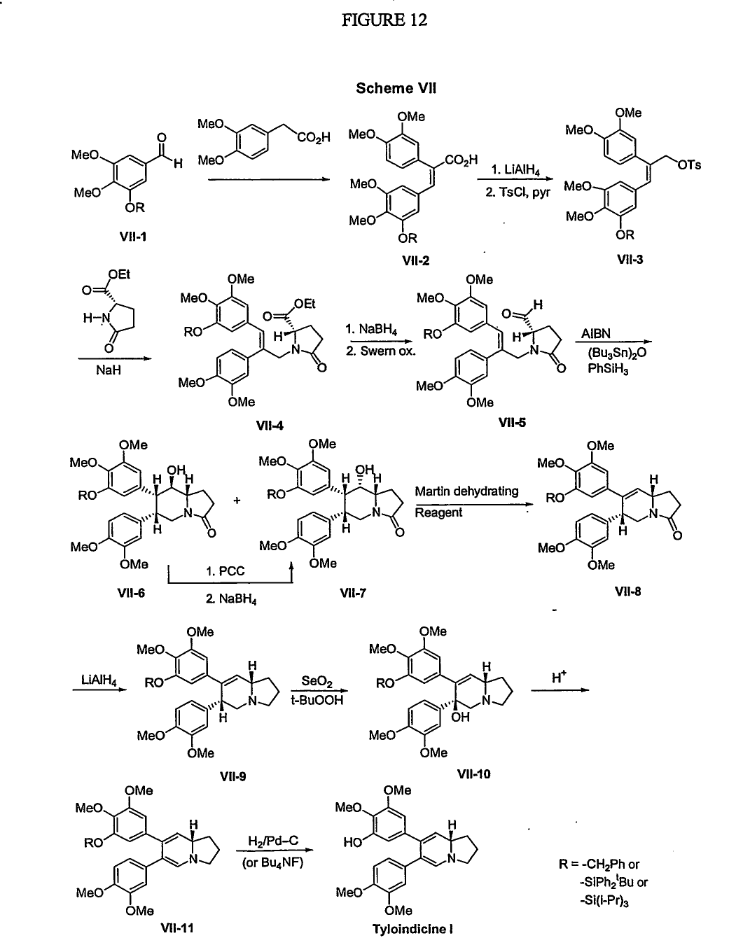

(d) Synthesis of tyloindicine I Inasmuch as tylo I has a free phenolic OH function, a protective group is necessary, as shown in FIG. 12, Scheme VII. A robust protective group, e.g., benzyl is preferred. Attempted hydrogenation of II-2 demonstrates that the double bond does not reduce under neutral conditions under 1 atm H₂ with Pd—C. Therefore, problems with removal of the benzyl group are minimal. However, if desired, an alternative scheme is to use t-BuPh₂Si or (i-Pr)₃Si protection, which are removable with F. (Alternatively, benzyl can be cleaved under any of several nonhydrogenolytic conditions.) Therefore, 3-benzyloxy-4,5-dimethoxybenzaldehyde (VII-1, Aldrich or preparation; or silyl-protected equivalent) is condensed with 3,4-dimethoxyphenylacetic acid to give the carboxylic acid VII-2. Reduction with LiAlH₄, followed by tosylation of the resulting alcohol, gives the tosylate VII-3. Displacement with ethyl (+)-(S)-2-pyrrolidine-5-carboxylate sodium salt then furnishes the adduct VII-4, which upon NaBH₄ reduction and Swern oxidation of the intermediate alcohol gives the aldehyde VII-5. Reductive cyclization then gives a mixture of alcohols VII-6b and VII-7. The stereoselectivity of the reaction may be assessed by NMR spectroscopy and by HPLC. The stereochemistry may be determined by X-ray crystallography. Separation of diastomers may be carried out by chromatography. VII-6 is converted to VII-7 by sequential PCC oxidation-NaBH₄ reduction.

The Martin sulfurane dehydrating reagent then provides the alkene VII-8 of defined stereochemistry. LiAlH₄ reduction, followed by SeO₂-t-BuOOH hydroxylation as per the example in Scheme II (conversion of II-2 to II-3), then gives the benzylic alcohol VII-10. The relative configuration of the compound is determined by 1D and 2D NMR spectroscopy, and by X-ray crystallography if a suitable crystal is available. Dehydration should prove facile by treatment of VII-10 with acid. Hydrogenolysis (H₂/Pd—C) (or for silyl groups, Bu₄NF) then provides the target tylo I. The compound may be thoroughly characterized by MS and NMR spectroscopy.

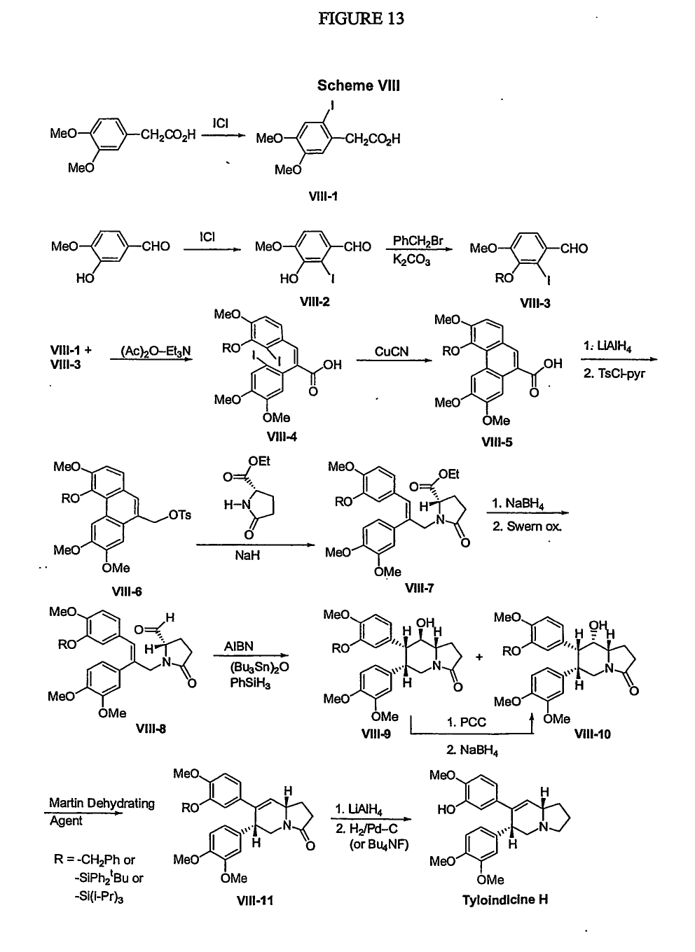

(e) Synthesis of tyloindicine H. Owing to its unsymmetrical substitution pattern on the aryl groups, tyloindicine H requires a more directed approach for the phenanthrene ring closure (FIG. 13, Scheme VIII). The results from some limited preliminary studies indicated that the VOF₃ closure on a non-iodinated version of VIII-4 leads to the wrong isomer. Thus 3,4-dimethoxyphenylacetic acid is ortho-iodinated using iodinemonochloride (ICl) to give 2-iodo-4,5-dimethoxyphenylacetic acid VIII-1. Similarly, iodination of 3-hydroxy-4-methoxybenzaldehyde gives 3-hydroxy-2-iodo-4-methoxybenzaldehyde VIII-2, which is then benzylated (or silylated with t-BuPh₂SiCl or (i-Pr)₃SiCl) to give VIII-3. Condensation of VIII-1 with VIII-3 under conditions used in the previous examples furnishes the carboxylic acid VIII-4. Ring closure via Ullman-type coupling using CuCN or Pd(PPh₃)₄ then furnishes the phenanthrene carboxylic acid VIII-5. Reduction with LiAlH₄ and tosylation of the intermediate alcohol gives the tosylate VIII-6. Displacement of the tosylate with ethyl (+)-(S)-2-pyrrolidine-5-carboxylate sodium salt then gives adduct VIII-7. Reduction with NaBH₄, followed by Swern oxidation of the intermediate alcohol, then gives the aldehyde VIII-8. Reductive cyclization follows closely the results of that for the tylo G synthesis (Scheme I), giving the correct stereosystem as shown for compounds VIII-9 and VIII-10. NMR studies, and an X-ray crystal structure if possible, may be used to establish structure. Use of the Martin sulfurane dehydrating agent, followed by LiAlH₄ reduction and removal of the protecting group, then furnishes tyloindicine H.

### Synthesis of Tyloindicine Analogues

(a) General Considerations Based on findings that analogues II-3 (NSC-716802) and II-2 (NSC-717335), as well as tylo H and I, are active in a sixty-panel in vitro screen against human-derived tumors, it is conceived that the tyloindicines are quite tolerant of modification, not only in the aromatic system, but also in the indolizidine system. The fact that the nonhydroxylated indolizidines tylos H and I, as well as II-2, are active, lends support to the notion that the OH group is not absolutely necessary for potent antitumor activity, but may serve to increase activity beyond GI₅₀'s of about 10⁸ M. The tyloindicine analogues lacking the OH group are chemically more stable than the hemiaminals, tylos F and G.

Two basic types of modifications on the tyloindicines may be made: (1) modification in the indolizidine ring system and (2) modifications on the aromatic system. The former has a profound effect on the activity, including the spectrum of activity against a range of tumors. The latter will serve to alter log P and other related parameters that might figure importantly in issues of solubility and drug disposition and delivery.

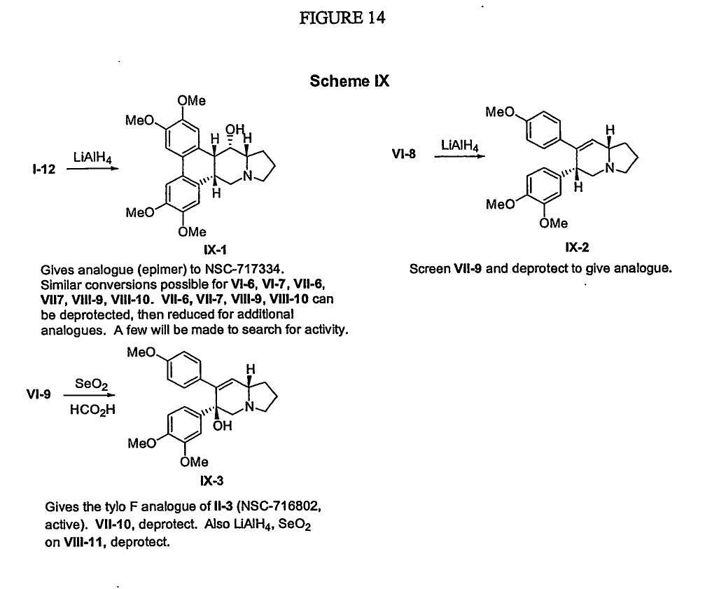

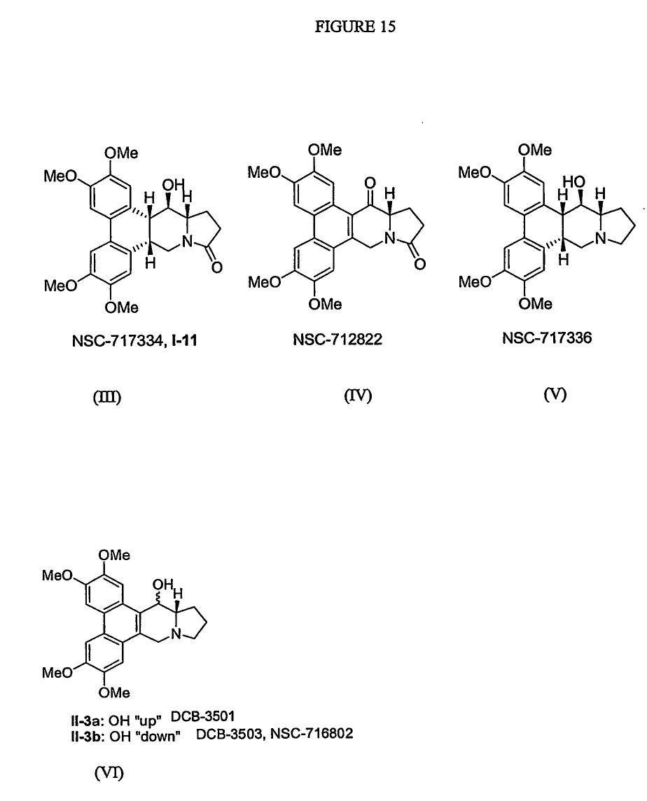

(b) Synthesis of Congeners in the Tyloindicine Series FIG. 14, Scheme A, shows a list of exemplary congeners that can be readily obtained by one- or two-step processes from the routes developed for the lead compounds.

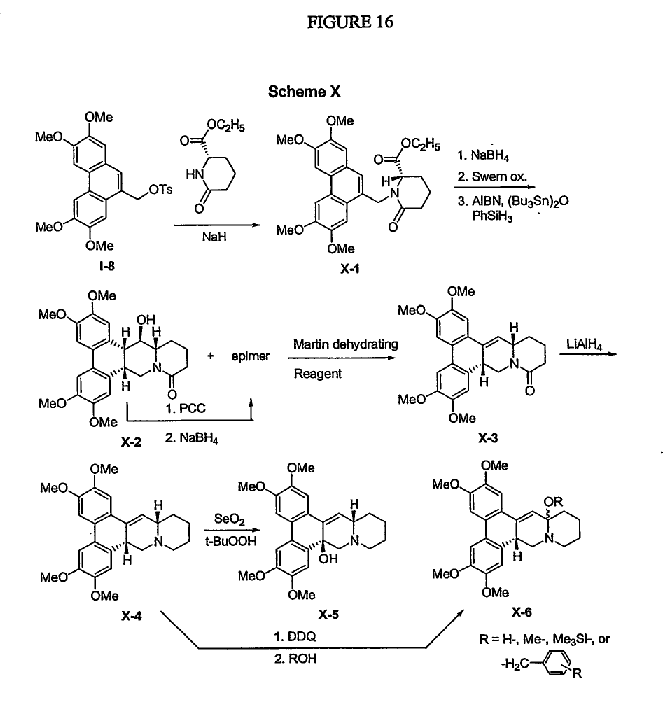

(c) Synthesis of Quinolizidine Analogues of Tyloindicine G A family of quinolizidine alkaloids have been synthesized as their racemic mixtures by a Diels-Alder route; however, none of these have apparently been screened for antitumor activity. The synthesis outlined in FIG. 16, Scheme X may be carried out. The requisite ethyl (s)-5-oxo-piperidine-2-carboxylate is expensive to synthesize. The racemic compound (Aldrich) may be used to develop the synthesis, and if active compounds emerge, then shift to the optically active material. Since the fused six-membered ring systems may behave differently from the fused 5,6-systems of the tyloindicine syntheses, altered procedures may be necessary. While the initial condensation to give X-1 goes as with the earlier examples, the reductive ring closure gives different isomeric mixtures of condensed alcohols X-2+epimer; configurations are easily recognized by simple IR C:H stretches, called Bohlmann bands. Wenkert, E.; Roychaudhari, D. K. The C-3 configuration of certain indol alkaloids. J. Am. Chem. Soc. 1956, 78, 6417-6418. An X-ray crystal structure may be obtained. Also, the DDQ deprotonation behaves differently, as an imminium species as formed in X-4, giving perhaps a more stabilized entity. Analogues of the other tyloindicines may be similarly synthesized.

Supporting Syntheses

(a) Supporting Syntheses

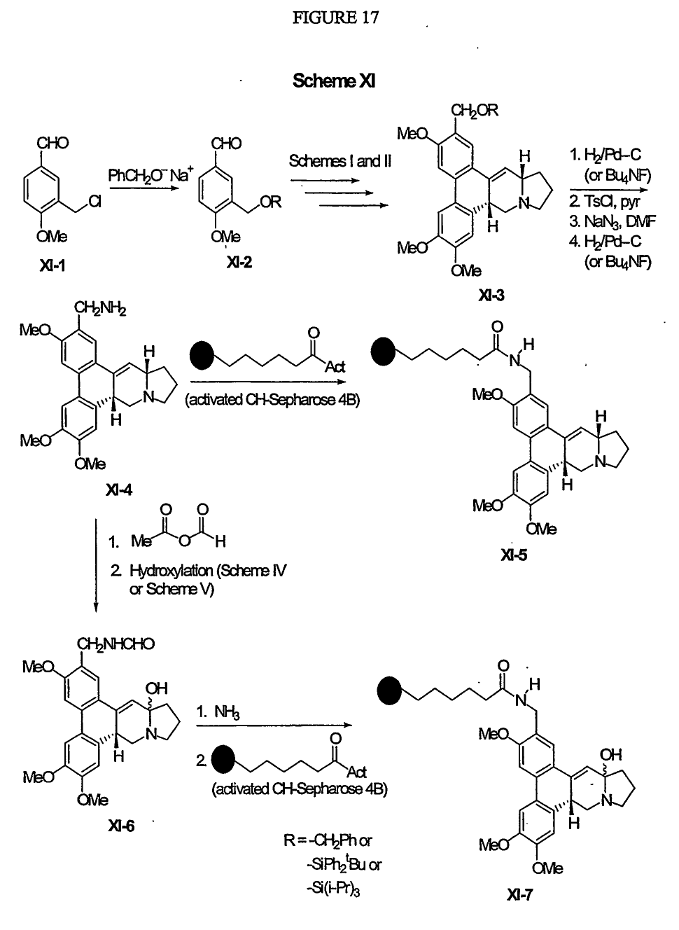

(a) Synthesis of Ligands for Affinity Chromatography For experiments designed to isolate the proteins that interact with the active drugs, active compounds with a reactive amino group for attaching to CH-Sepharose 4B are desired, as the tertiary OH groups are not useful for such conjugation; the phenolic OH groups of tylos H and I may be functional, but an amino group would be preferred. Synthesis of an analogue of compound II-2 (NSC-717335) is shown in FIG. 17, Scheme XI. Thus 3-chloromethyl-4-methoxybenzaldehyde XI-1 is reacted with sodium benzylate to give 3-benzyloxymethyl-4-methoxybenzaldehyde XI-2; alternatively silyl protection may be used. Using this protected alcohol in the processes outlined in Schemes I and II, one may synthesize XI-3, which is the benzyloxy- (or silyloxy-) methyl analogue of II-2. Using the sequence of benzyl deprotection via hydrogenolysis (or any one of a number of other methods) (or Bu₄NF for silyl) tosylation, azide displacement, and reduction (by hydrogenation or reaction with Ph₃P), the aminomethyl analogue XI-4 can be obtained. Reaction with activated CH-Sepharose (said by Pharmacia to be an active ester) then provides the Sepharose drug conjugate XI-5. Compound x[4, for example, may be examined for antitumor activity.

The tylo G analogue can be prepared by protecting the amino function of XI-4, then carrying out the chemistry in Schemes IV or V. The formyl group can function as a protective group, as it is removable in acid or base and withstands the DDQ reagent of Scheme IV or the triflation step of Scheme V. The synthesis proceeds as shown in Scheme XI (XI-4; XI-7). Alternatively the azido derivative from XI-3 may be hydroxylated, the product reduced, and then subjected to conjugation with activated CH-Sepharose. The other analogues (tylos H and I) as well as any active compounds synthesized as congeners can be similarly modified for immobilization on Sepharose.

An alternate sequence can lead to tethered analogues of DCB-3500, -3501 and -3503. Either use of an amino group or a selectively protected OH on the aryl ring could serve as an anchor for connecting or synthesizing the tether to the basic molecule.

(b) Synthesis of Compounds for Radiolabeling Radiolabeling can be carried out by either of two procedures: (1) catalytic exchange labeling with ³H₂ on the final product, or (2) by carrying out the amide reduction step with LiAl³H₄, then hydroxylating for tylos F and G. The exchange reaction may be the method of choice for tylos H and I that do not have the sensitive hemiaminal function, while the more laborious two-step procedure is more useful for tylos F and G; however, rearrangements in any of the compounds are possible. Active congeners that are selected for in-depth studies may be evaluated for radiolabeling.

The following represents an experimental writeup of the chemical syntheses which are set forth in FIGS. 6 and 8 of the present invention.

### Total synthesis of (+)-(S)-Tylophorine (FIGS. 6 and 8)

Experimental Section

### 3,4-Dimethoxyphenylacetic acid (I-1)

3,4-Dimethoxyphenylacetonitrile (12.1 g, 68.0 mmol) and sodium hydroxide (7.1 g, 178 mmol) were dissolved in a mixture of water (21 mL) and ethanol (10 mL) and heated under reflux for 10 h. The solution was cooled to room temperature, diluted with water (50 mL) and extracted with ether (3×40 mL). Dissolved ether was removed from the aqueous layer in vacuo. Acidification of the aqueous ether-free solution with dilute hydrochloric acid produced a white precipitate. The suspension was cooled to 4° C., and the precipitate was collected by filtration to yield I-1 (11.8 g, 88.6%): mp 97-99° C.

### 2,3-Bis-(3,4-dimethoxyphenyl)acrylic acid (I-3)

Veratraldehyde (I-2)(15.6 g, 94.0 mmol), acid I-1 (20.0 g, 104 mmol), acetic anhydride (40 mL), and triethylamine (20 mL) were heated together at 100° C. for 24 h with the exclusion of moisture. The solution was allowed to cool to room temperature, water (100 mL) was added, and the mixture was stirred for 1 h. The mixture was then poured into aq potassium carbonate (75 g in 250 mL) and refluxed until nearly all the gummy material had dissolved. The solution so obtained was cooled, extracted with ether (2×50 mL), and carefully acidified with concentrated hydrochloric acid (pH 5) to produce a white precipitate. The solid that separated was collected and recrystallized from methanol to give I-3 (11.2 g, 68%.). ¹H NMR (300 MHz, CDCl₃): δ 7.67 (s,1H), 6.56-6.69 (m, 6H), 3.90 (s, 3H), 3.65 (s, 3H), 3.62 (s, 31), 3.46 (s, 3H).]³C NMR (75 MHz, CDCl₃): δ 168.42, 149.78, 149.11, 148.42, 148.00, 140.23, 129.50, 128.62, 127.30, 125.18, 122.10, 112.80, 112.36, 111.38, 110.36, 55.94, 55.78, 55.24, 52.35.

### Methyl 2,3,-bis-(3,4-dimethoxyphenyl)acrylate (I-4)

2,3-Bis-(3,4-dimethoxyphenyl)acrylic acid I-3 (3.44 g, 10.0 mmol) was dissolved in a solution of 1.5% concd sulfuric acid in anhydr methanol (150 mL), and the resulting solution was heated to reflux for 10 h. After evaporating the solvent under reduced pressure, chloroform (100 mL) and water (50 mL) were added to the residual oil. The organic phase was separated, and the aq phase was extracted with chloroform (2×30 mL). The combined organic phase was washed with 10% NaHCO₃ (50 mL), water (40 mL), brine, and dried over Na₂SO₄. The solvent was evaporated to afford product I-4 (3.41 g, 95.3%). ¹H NMR (250 MHz, CDCl₃): δ 7.77 (s, 1H), 6.52-6.69 (m, 6H), 3.90 (s, 3H), 3.84 (s, 3H), 3.81 (s, 3H), 3.79 (s, 1H), 3.46 (s, 3H).

### Methyl 2,3,6,7-Tetramethoxyphenanthrene-9-carboxylate (I-5)

To a chilled solution of I-4 (7.2 g, 20 mmol) in dry CH₂Cl₂ (400 mL) was added trifluoroacetic acid (60 mL) followed by vanadium(V) oxytrifluoride (7.2 g, 6.00 mmol). After stirring for 2 days at 5° C., the reaction mixture was quenched with 1 M aq citric acid, and the organic layer was washed with 1 M aq citric acid (3×120 mL) and brine. The organic layer was dried (Na₂SO₄), and filtered through a short silica gel column to give upon evaporation of the solvent ester I-5 (6.72 g, 94.3%). ¹H NMR (300 MHz, CDCl₃) δ 8.65 (s, 1H), 8.42 (s, 1H), 7.79 (s, 1H), 7.75 (s, 1H), 7.26 (s, 1H), 4.14 (s, 3H), 4.13 (s, 3H), 4.08 (s, 3H), 4.04 (s, 3H), 4.02 (s, 3H). ¹³C NMR (CDCl₃, 75 MHz) δ 168.36, 151.38, 149.41, 149.14, 130.14, 127.29, 125.30, 124.71, 124.37, 122.34, 109.44, 107.03, 102.85, 102.65, 56.37, 56.26, 56.22, 56.15, 52.36.

### (2,3,6,7-Tetramethoxyphenathren-9-yl)methanol (I-6)

To a cooled suspension of lithium aluminum hydride (2.70 g, 70.0 mmol) in dried THF (100 mL) was added dropwise during 30 min a solution of I-5 (3.56 g, 1.00 mmol) in dry THF (200 mL), which was maintained under a nitrogen atmosphere. The reaction mixture was allowed warm to room temperature for 4 h, then cooled to 0° C., at which temperature ethyl acetate (100 mL) and 2 N hydrochloric acid (70 mL) were added. The precipitate was filtered and washed with ethyl acetate. The filtrate was concentrated, and the residual oil was purified by flash column chromatography (4:1 CH₂Cl₂-EtOAc) to afford I-6 (6.04 g, 91.6%): ¹H NMR (300 MHz, CDCl₃) δ 7.72 (s, 1H), 7.66 (s, 1H), 7.46 (s, 1H), 7.08 (s, 1H), 5.04 (s, 2H), 4.09 (s, 3H), 4.07 (s, 3H), 4.01 (s, 3H), 3.96 (s, 3H). ¹³C NMR (75 MHz, CDCl₃) δ 148.95, 148.63, 148.43, 148.37, 131.82, 125.57, 124.72, 124.34, 124.24, 123.61, 108.10, 104.56, 102.93, 102.46, 64.50, 55.93, 55.90, 55.82, 55.74.

### 2,3,6,7-Tetramethoxyphenanthren-9-yl)methyl p-toluenesulfonate (I-7)

To an ice-cold, stirred solution of alcohol I-6 (570 mg, 1.86 mmol) and triethylamine (210 mg, 2.08 mmol) in CH₂Cl₂ (10 mL) was added p-toluenesulfonyl chloride (400 mg, 2.05 mmol) in CH₂Cl₂ (6 mL). The reaction mixture was stirred for 10 min at room temperature. Water (20 mL) was added to the mixture, the organic layer was separated and washed with saturated NaHCO₃, water, and brine, and dried over Na₂SO₄. The solvent was removed in vacuo to give a residue that was purified by silica gel column chromatography (100:2 CH₂Cl₂—H₃OH) to afford I-7 (737 mg, 88%) that was directly used in the next step.

### Ethyl (S)-1-Oxo-1-(2,3,6,7-tetramethoxyphenathren-9-ylmethyl)pyrrolidine-2-carboxylate (I-8)

A solution of ethyl (S)-(+)-2-pyrrolidone-5-carboxylate (408 mg, 2.68 mmol) in DME (10 mL) was added dropwise to a stirred suspension of sodium hydride (62 mg, 2.6 mmol) in DME (6 mL) under N₂ at ice-bath temperature. When all of the sodium hydride had reacted, tosylate I-7 (1.10 g, 2.28 mmol) was added, and the reaction mixture was heated for 72 h at 70° C. After evaporation of most of the solvent, the residue was saponified by refluxing in 2 N ethanolic potassium hydroxide (20 mL) overnight. The reaction mixture was cooled to room temperature, chloroform (100 mL) was added, and the organic layer was washed with water, 1 N HCl and brine, and dried over Na₂SO₄. The solution was evaporated, the residual oil was purified by column chromatography (6:1 CH₂Cl₂-EtOAc) to give I-8 (706 mg, 66.3%): mp 185-186° C., [α]_{D}²²+113.8° (c 1.0, CH₂Cl₂). IR (KBr) 3447, 2930, 2849, 1737, 1687, 1513, 1476, 1435, 1258, 1201, 1150, 1064, 1636, 774 cm⁻¹. ¹H NMR (300 MHz, CDCl₃) δ 7.82 (s,1H), 7.79 (s,1H), 7.63 (s,1H), 7.42 (s,1H), 7.17 (s,1H), 5.53 (d, J=14.7 Hz), 4.42 (d, J=14.4 Hz, 1H), 4.13-3.98 (m, 14H), 3.82 (dd, J=4.2 Hz, J=9.3 Hz, 1H), 2.68-2.56 (m, 1H), 2.45-2.35 (m, 1H), 2.20-1.95 (m, 2H), 1.18 (m, 3H). ¹³CNMR (75 MHz, CDCl₃) δ 174.46, 171.60, 149.44, 149.04, 148.95, 148.76, 127.07, 126.82, 125.46, 124.78, 108.10, 105.22, 103.04, 102.67, 61.34, 58.68, 56.36, 56.13, 56.05, 55.95, 44.79, 29.93, 22.83, 14.2. ESIMS Calcd for C₂₈H₂₉NO₇ (M) 467.19. Found 467.193.

### (S)-5-Hydroxymethyl-1-(2,3,6,7-tetramethoxyphenanthren-9-ylmethyl)pyrrolidin-2-one (I-9)

To a solution of I-8 (6.70 g, 14.0 mmol) in TBF (150 mL) and ethanol (400 mL) was added NaBH (2.06 g, 55.8 mmol) at room temperature. After stirring 60 h at room temperature, concd HCl (1 mL) was added, the mixture was stirred for 1 h, the solvents were evaporated, and the residual oil was then purified by flash column chromatography to give I-8 (5.53 g, 92.7%): mp 236-237° C. [α]_{D}²²+97.6° (c 1.0, CH₂Cl₂) IR (KBr) 3434, 2936, 2835, 1727, 1662, 1622, 1512, 1475, 1435, 1256, 1199, 1149, 1064, 1038, 840, 773 cm⁻¹. ¹HNMR (300 MHz, CDCl₃) δ 7.77 (s, 1H), 7.73 (s, 1H), 7.55 (s, 1H), 746 (s, 1H), 7.14 (s, 1H), 5.39-5.35 (d, J=12 Hz,1H), 5.45-4.55 (dd, J=15 Hz, 1H), 4.09 (s, 1H), 3.78 (m,1H), 3.49 (m, 2H), 2.70-2.65 (m, 1H), 2.47-2.08 (m, 1H), 1.92 (m, 2H). ¹³C NMR (75 MHz, CDCl₃) δ 175.62, 149.43, 149.04, 148.96, 148.78, 127.48, 126.35, 125.41, 124.87, 124.64, 124.43, 108.05, 104.92, 103.14, 102.65, 62.57, 58.64, 56.39, 56.12, 56.01, 55.93, 44.51, 30.75, 21.27. ESIMS Calcd for C₂₄H₂₇NO₆ (M+) 425.2. Found 425.1842.

### (S)-5-Oxo-1-(2,3,6,7-tetramethoxyphenathren-9-ylmethyl)pyrrolidine-2-carbaldehyde (I-10)

To oxalyl chloride (2.2 mL, 25 mmol) in CH₂Cl₂ (25 mL) at −78° C. under argon was added DMSO (3 mL, 52 mmol) in CH₂Cl₂ (15 mL). The mixture was stirred for 5 min, and then alcohol I-9 (5.1 g, 12 mmol) in CH₂Cl₂ (220 mL) was added over a 10 min period. The reaction mixture was stirred at −78° C. for 30 min, then triethylamine (24.6 mL, 176.4 mmol) was added with stirring for 20 min. The mixture was allowed to warm to room temperature for 10 min, and then it was poured into a separatory funnel containing water (100 mL). The organic layer was separated, and the aq layer was extracted with dichloromethane (2×50 mL). The combined organic layers were washed with 1% HCl (50 mL), satd NaHCO₃ (60 mL), water, and brine and dried over anhydrous MgSO₄. The solvents were evaporated, and the residue was purified by column chromatography, eluting with (8:1 CH₂Cl₂-EtOAc) to afford aldehyde I-10 (5.07 g, 96%) as a slightly yellow solid: mp 208-210° C. [α]_{D}²²+56.7° (c 1.00 CHCl₃) IR (KBr) 3404, 2937, 1729, 1665, 1621, 1512, 1475, 1435, 1256, 1199, 1149, 1064, 1038, 841, 773 cm⁻¹. ¹H NMR (300 MHz, CDCl₃) δ 9.20 (s, 1H), 7.80 (s, 1H), 7.75 (s, 1H), 7.60 (s, 1H), 7.38 (s, 1H), 7.15 (s, 1H), 5.34-4.29 (d, J=15 Hz,1H), 4.73-4.68(d, J=15 Hz, 1H), 4.11 (s, 3H), 4.09 (s, 3H), 4.03 (s, 3H), 4.02 (s, 3H), 3.86-3.80 (m, 1H), 2.56-2.38 (m, 2H), 2.18-1.89 (m, 2H). ¹³C NMR (75 MHz, CDCl₃) δ 198.37, 174.42, 149.55, 149.07, 148.98, 148.79, 127.10, 126.64, 125.32, 124.88, 124.77, 124.44, 108.14, 104.95, 103.12, 102.59, 64.50, 56.34, 56.01, 55.93, 45.32, 29.68, 19.23. ESIMS Calcd for C₂₄H₂₇NO₆ (M) 425.2. Found m/z 425.1842.

### (8bR,12aS,13R,13aS)-13-Hydroxy-2,3,6,7-tetramethoxy-8b,11,12,12a,13,13a-hexa hydro-9H-9a-aza-cyclopenta[b]triphenylen-10-one (I-11) and (8bR,12aS,13S,13aS)-13-Hydroxy-2,3,6,7-tetramethoxy-8b,11,12,12a,13,13a-hexahydro-9H-9a-aza-cyclopenta[b]triphenylen-10-one (I-12)

To a solution of I-10 (2.12 g, 5.00 mmol) in dry benzene (15 mL) in a 100-mL sealed Schlenk tube was added (Bu₃Sn)₂O (38 μL, 0.75 mmol), PhSiH₃ (31.0 μL, 2.50 mmol), EtOH (585 μL, 2.00 mmol), and AIBN (90 mg, 2.5 mmol in benzene (2.0 mL). The vessel was sealed, shaken, and placed in an oil bath at 80-85° C. After 12 h, TLC analysis indicated that all of the starting material had been consumed. The mixture was allowed to cool to room temperature, and tetrabutylammonium fluoride (30.0 mL of a 1.0 M solution in THF, 3.0 mmol) was added with stirring for 2 h, at the end of which time 15 mL of 2 N HCl was added. The reaction mixture was extracted with CH₂CH₂ (3×50 mL), and the combined organic extracts were dried (MgSO₄), filtered, and concentrated. The residue was purified by flash chromatography (eluting with 3:1:0.01 CH₂H₂-EtOAc—CH₃OH) to give 827 mg (68.8%) of compound I-11: [α]_{D}²²+78.3° (c 0.48, CHCl₃); IR(KBr) 3397, 2937, 1660, 1607, 1510, 1464, 1406, 1267, 1249, 1202, 1406, 1267, 1249, 1202, 1014, 770 cm⁻¹. ¹H NMR (300 MHz, CDCl₃): δ 7.19 (s, 1H), 7.11 (s, 1H), 7.09 (s, 1H), 6.82 (s, 1H), 4.86 (d. J=14 Hz, 1H), 3.98 (s, 3H), 3.95 (s, 3H), 3.94 (s, 3H), 3.92 (s, 3H), 3.41(dd, J=7.5 Hz, J=16.2 Hz, 1H), 3.30 (t, 1H), 3.12 (d, J=14.1 Hz, 1H), 3.03 (t, 1H), 2.69 (dd, J=4.8 Hz, J=9.6 Hz, 1H), 2.44-2.21(m, 3H), 1.69-1.59 (m, 1H). ¹³C NMR (75 MHz, CDCl₃): 173.50, 148.81, 148.47, 147.76, 147.67, 127.15, 126.47, 126.30, 126.01, 112.70, 108.95, 107.26, 107.27, 71.85, 61.51, 56.30, 56.19, 56.11, 56.08, 48.20, 39.84, 37.39, 30.15, 22.49. HRMS Calcd for C₂₄H₂₇NO₆ (M+) 425.2. Found 425.1842. Compound I-12: (30%). [α]_{D}²²−104.2° (c 1.0, CHCl₃). IR (KBr) 3434, 2934, 1681, 1606, 1509, 1462, 1407, 1268, 1204, 1119, 1039, 1024, 1007, 858, 778 cm⁻¹. ¹H NMR (300 MHz, CDCl₃) δ 7.30 (s, 1H), 7.20 (s, 1H), 7.13 (s, 1H), 6.75 (s, 1H), 5.02-4.97(d, J=15 Hz, 1H), 3.98 (s, 3H), 3.94 (s, 3H), 3.91(s, 3H), 3.79 (s, 3H), 3.78 (m, 2H), 3.24 (t, 1H), 3.11-3.17(m, 1H), 2.98-3.00 (m, 1H), 2.33 (t, 2H), 2.05-2.11 (m, 2H). ¹³C NMR (75 MHz, CDCl₃) δ 174.20, 148.84, 148.79, 148.66, 148.07, 128.47, 127.57, 127.06, 126.00, 110.98, 108.58, 107.33, 106.63, 73.31, 61.57, 56.29, 56.26, 56.07, 44.60, 39.83, 34.50, 30.28, 18.54. HRMS Calcd for C₂₄H₂₇NO₆ (M+) 425.2. Found 425.1842.

### (8bR,12aS,13R,13aS)-2,3,6,7-Tetramethoxy-10-oxo-8b,9,10,11,12,12a,13,13a-octahydro-9a-aza-cyclopenta[b]triphenylen-13-yl methanesulfonate (III-1)

To an ice-cold, stirred solution of alcohol I-11 (1.06 g, 2.50 mmol) and triethylamine (950 mg, 9 mmol) in CH₂Cl₂ (35 mL) was added methanesulfonyl chloride (800 mg, 6 mmol) in CH₂Cl₂ (3 mL). The reaction mixture was stirred for 10 min at room temperature. Water (20 mL) was added to the mixture, and the organic layer was separated, washed with satd NaHCO₃, water, and brine, and dried (Na₂SO₄). The solvent was evaporated to give a residue, that was purified by silica gel column chromatography (100:2 CH₂Cl₂—H₃OH) to afford 11 (1.25 g, 100%). mp 222-224° C. [α]_{D}²²+98.3° (c 0.48, CHCl₃). IR (KBr): 3438, 2935, 1688, 1607, 1566, 1511, 1464, 1410, 1348, 1239, 1204, 1174, 1118, 957, 832, 770, 699 cm⁻¹. ¹H NMR (300 MHz, CDCl₃) δ 7.16 (s, 1H), 7.15(s, 1H), 7.04 (s, 1H), 6.82 (s, 1H), 5.28 (s,1H), 4.86 (d, J=18 Hz, 1H), 3.98 (s, 3H), 3.97 (s, 3H), 3.93 (s, 3H), 3.29 (s, 3H), 3.64 (q, 1H), 3.36 (br, 1H), 3.12 (dd, J=2.7 Hz, J=18 Hz, 1H), 2.95 (dd, J=4.2 Hz, J=10.2 Hz, 1H), 2.33 (m, 3H), 1.98 (m, 1H). ¹³C NMR (75 MHz, CDCl₃) δ 174.15, 149.69, 149.24, 148.50, 148.02, 127.13, 126.40, 126.06, 125.46, 113.56, 109.17, 107.63, 82.18, 61.16, 56.57, 56.24, 46.47, 39.96, 38.45, 38.05, 30.12, 22.34. ESIMS Calcd for C₂₄H₂₇NO₆ (M+) 503.16, Found 503.163.

### 2,3,6,7-Tetramethoxy-8b,11,12,12a-tetrahydro-9H-9a-aza-cyclopenta[b]triphenylen-10-one (III-2)

A solution of methanesulfonate III-1 (350 mg, 0.8 mmol) and potassium tert-butoxide (116 mg, 1.3 mmol) in DMSO (5 mL) was stirred at room temperature for 6 h. Water (5 mL) and ethyl acetate (20 mL) were added, the organic layer was separated, and the aq layer was extracted with ethyl acetate (3×20 mL). The organic extract was washed with water and brine and dried over Na₂SO₄. The solvent was evaporated to give a solid residue that was purified by silica gel column chromatography to afford III-2 (236 mg, 82.6%). mp 252-254° C., [α]_{D}²²+108° (c 0.25, CHCl₃). IR (KBr): 3438, 2933, 2837, 1680, 1620, 1514, 1468, 1424, 1249, 1211, 1148, 1044, 774, 699 cm⁻¹. ¹H NMR (300 MHz, CDCl₃) δ 7.82 (s, 1H), 7.81 (s, 1H), 7.27 (s, 1H), 7.16 (s, 1H), 5.32 (d, J=16.2 Hz, 1H), 4.50 (d, J=16.5 Hz, 1H), 4.11 (s, 3H), 4.10 (s, 3M), 4.04 (s, 3H), 4.03 (s, 3H), 3.93 (m, 1H), 3.47 (dd, J=6 Hz, J=15.9 Hz, 1H), 3.86 (t, 1H), 2.62-2.51 (m, 3M), 2.205-1.99 (m, 1H). ¹³C NMR (75 MHz, CDCl₃) δ 173.96, 148.83, 148.63, 124.88, 124.30, 123.43, 123.36, 122.71, 103.66, 103.28, 103.23, 102.68, 56.06, 55.90, 53.22, 41.13, 33.56, 30.24, 25.37. ESIMS Calcd for C₂₄H₂₅NO₅ (M) 407.17, Found 407.173.

### (+)-(s)-Tylophorine

To a stirred solution of lithium aluminum hydride (50 mg, 1.35 mmol) in TBF (5 mL) was added a solution of III-2 (110 mg, 0.27 mmol) in THF (15 mL) at ice-bath temperature. The reaction mixture was allowed to warm to room temperature and stirred 4 h. Ice-water, EtOAc (5 mL), CH₂Cl₂ (20 mL) and saturated NH₄Cl (0.5 mL) were added, and the mixture was stirred for 1.5 h. It was then filtered through a pad of Celite, and the solvent was removed under reduced pressure. The residue was chromatographed on Al₂O₃ (100: 0-1.5 CH₂Cl₂ CH₃OH) to give (+)-(S)-tylophorine (94 mg, 88.6%): mp (230° C. dec) melt 260-261° C.; [α]_{D}²²+⁴⁹° (c 0.475, CHCl₃). IR (KBr): 3435, 2960, 1619, 1513, 1470, 1425, 1247, 1211, 1198, 1151.0 cm⁻¹. ¹H NMR (300 MHz,CDCl₃) δ 7.82 (s, 2H), 7.31 (s, 1H), 7.15 (s, 1H), 4.63 (d, J=14.4 Hz, 1H), 4.12 (s, 6H), 4.06 (s, 6H), 3.68 (d, J=14.4 Hz, 1H), 3.49 (t, J=8.4 Hz, 1H), 3.39 (d, J=15.3 Hz, 1H), 2.94 (t, J=12.3 Hz, 1H), 2.47-2.53 (m, 2H), 2.22-2.30 (m, 1H), 1.95-2.09 (m, 2H), 1.76-1.85 (m, 1H). ¹³C NMR (75 MHz,CDCl₃) δ 148.51, 148.31, 148.23, 126.12, 125.65, 124.16, 123.49, 123.29, 103.83, 103.28, 103.16, 102.97, 60.21, 56.03, 55.92, 55.87, 55.12, 53.94, 33.17, 31.28, 21.68. ESIMS Calcd for C₂₄H₂₇NO₄ (M) 393.19, found 393.194.

The following represents an experimental of the chemical syntheses which are set forth in FIG. 7 of the present invention.

### (S)-2,3,6,7-Tetramethoxy-8b,12,12a-tetrahydro-9H, 9a-aza-cyclopenta[b]-triphenylene-10-one (II-1)

Martin sulfurane dehydrating reagant, bis[α,α-bis(trifluoromethyl)benzenmethanolato]-diphenylsulfur (806 mg, 1.2 mmol) in CH₂Cl₂ (5 mL) was added to a solution of compound I-12 (255 mg, 0.6 mmol) in CH₂Cl₂ (6 mL) at −78° C. The reaction mixture was warmed to room temperature and stirred for 6 h. The solvent was removed under reduced pressure, and the residue was purified by flash chromatography (eluting with 6:2:0.2 CH₂Cl₂-EtOAc—CH₃OH) to afford product1 II-1 (99 mg, 81.6%) as a solid: mp 123-125° C.; [α]_{D}²²+108° (c 0.27, CHCl₃). IR (KBr) 3435, 2935, 2833, 1682, 1606, 1510, 1463, 1407, 1268, 1205, 1119, 1040, 858, 778 cm⁻¹. ¹HNMR (300 MHz, CDCl₃) δ 7.31 (s, 1H), 7.18 (s, 1H), 7.12 (s, 1H), 6.93 (s, 1H), 5.84 (d, J=1.2 Hz, 1H), 4.91(d, J=14.4 Hz, 1H), 4.34 (t, 1H), 3.99 (s, 6H), 3.94 (s, 3H), 3.93 (s, 3H), 3.52 (br, 1H), 3.12 (dd, J=5.4 Hz, J=14.8 Hz, 1H), 2.57-2.29 (m, 3H), 1.62-1.55 (m, 1H). ¹³C NMR (75 MHz, CDCl₃) 5172.28, 149.36, 148.23, 147.55, 136.80, 129.25, 128.50, 126.57, 126.45, 122.84, 107.95, 107.26, 106.73, 106.33, 56.41, 56.30, 56.19, 56.06, 54.99, 38.50, 36.27, 31.54, 25.81. HRMS Calcd for C₂₄H₂₇NO₅ (+407.17; Found 407.173. Anal. Calcd for C₂₄H₂₅NO₅—H₂O: C, 67.75; H, 6.40; N, 3.29. Found: C, 67.55; H, 6.05; N, 3.30.

### (S)-2,3,6,7-Tetramethoxy-8b,9,10,11,12,12a-hexahydro-9a-aza-cyclopenta[b]-triphenylene (II-2)

To a solution of compound II-1 (163 mg, 0.4 mmol) in THF (6 mL) at −78° C. was slowly added freshly prepared alane (AlH₃, 5.2 mL of a 0.25 M solution in TBF, 1.3 mmol), and the reaction mixture was allowed to warm to −20 to −15° C. with stirring for 2.5 h. The reaction mixture was again cooled to −50° C. and quenched with 5:95 water-TBF (3.5 mL). The solvent was then removed under pressure, and the residue was partitioned between 0.01 N NaOH (4.5 mL) and CH₂Cl₂ (25 mL). The aqueous layer was extracted with CH₂Cl₂ (3×10 mL), and the combined extracts were washed with brine (10 mL), dried over (Na₂SO₄), and concentrated under reduced pressure. The crude product was purified by flash chromatography on silica gel (eluting with 100:3:0.05 CH₂Cl₂—CH₃OH—NO₄H) to give compound H-2 (119 mg, 75.5%).

### (12S,13S)-2,3,6,7-Tetramethoxy-9,10,11,12,12a,13-hexahydro-9a-aza-cyclopenta[b]triphenylen-13-ol and tyloindicine G

Selenium dioxide (84 mg, 0.776 mmol) was added to a solution of compound II-2 (150 mg, 0.36 mmol) in dioxane (1 mL) and formic acid (99% purity, 2 mL) maintained at ice-bath temperature. After allowing the mixture to warm to room temperature and stir for 2.5 h, the mixture was diluted with water (5 mL) and CH₂Cl₂ (25 mL), and the insoluble material was filtered through a pad of Celite. The filtered solution was extracted with CH₂Cl₂ (2×20 mL). The organic layers were washed with satd Na₂S₂O₃ solution and satd NaCl, dried over anhydrous MgSO₄, filtered and concentrated under reduced pressure. The residue was chromatographed on a silica gel column (eluted with 100:3 CH₂Cl₂-CH₃OH) to give a mixture of II-3a and II-3b (102 mg, 60%), along with 50 mg (30%) of II-4 (tyloindicine G), both of which were identified by comparison of their NMR spectra with authentic materials.

Biological Studies

Compound II-3 (NSC-716802) was tested in in vitro cell culture studies. The NCI data is essentially confirmed with SK-MEL-2 (GI₅₀=0.16 μM) and SK-MEL-28 (GI₅₀=0.7 pn cell lines. In addition, potent activity was shown in two additional cell lines, KB (head and neck cancer) (GI₅₀=0.2 μM) and HepG2 (hepatocarcinoma) (GI₅₀=0.06 μM).

Several studies, including cross-resistance studies, clonogenic assays, and effect on cell cycle progression have been performed in cell culture with compound II-3 (NSC-716802). Toxicity and in vivo antitumor studies have been performed in mice with compound II-3 (NSC-716802). Comparative studies of growth inhibition of the two tyloindicine analogues, II-3 and II-2, have been performed in cell culture.

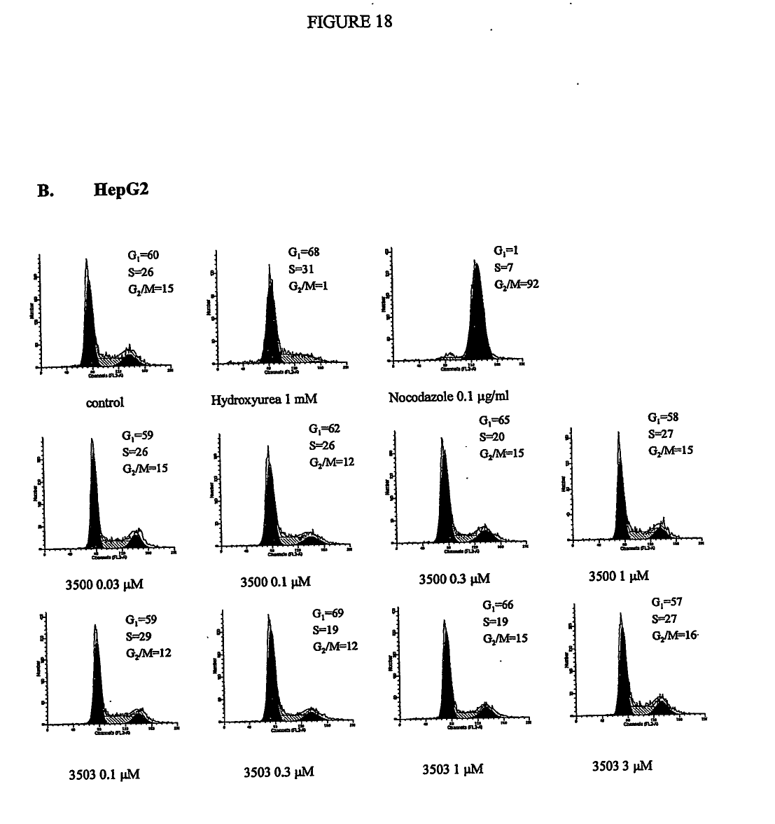

As shown in FIG. 18, Tables 1A and 1B, several KB and HepG2 cell lines were developed that are resistant to various anticancer drugs. The data show that cells that have become resistant to VP-16 (etoposide), VCR (vincristine), CPT (camptothecin), and DOX (doxorubicin) are sensitive to II-3 (referred to in the figures as ZH-152). These results further support the conception of the unique activity of II-3 and other tyloindicines and support the conception that the mode of action of II-3 (and other tyloindicines) differs from that of any of these anticancer drugs.

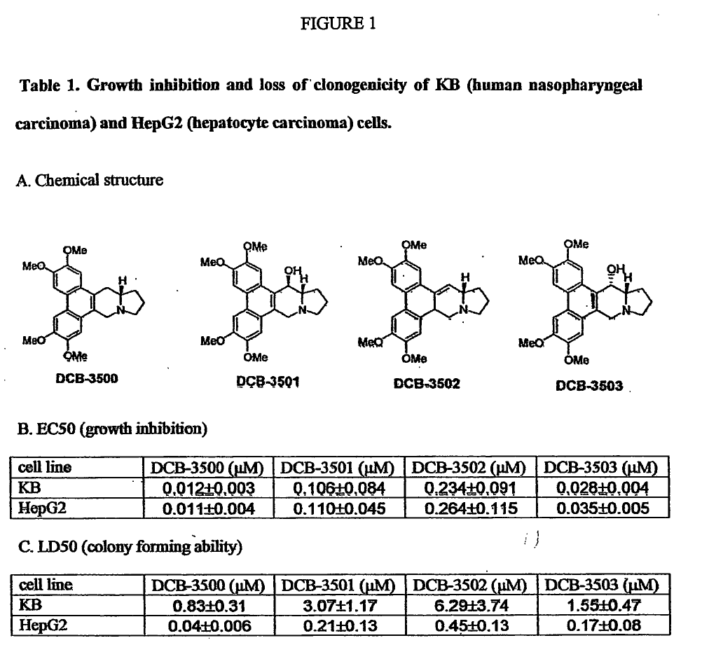

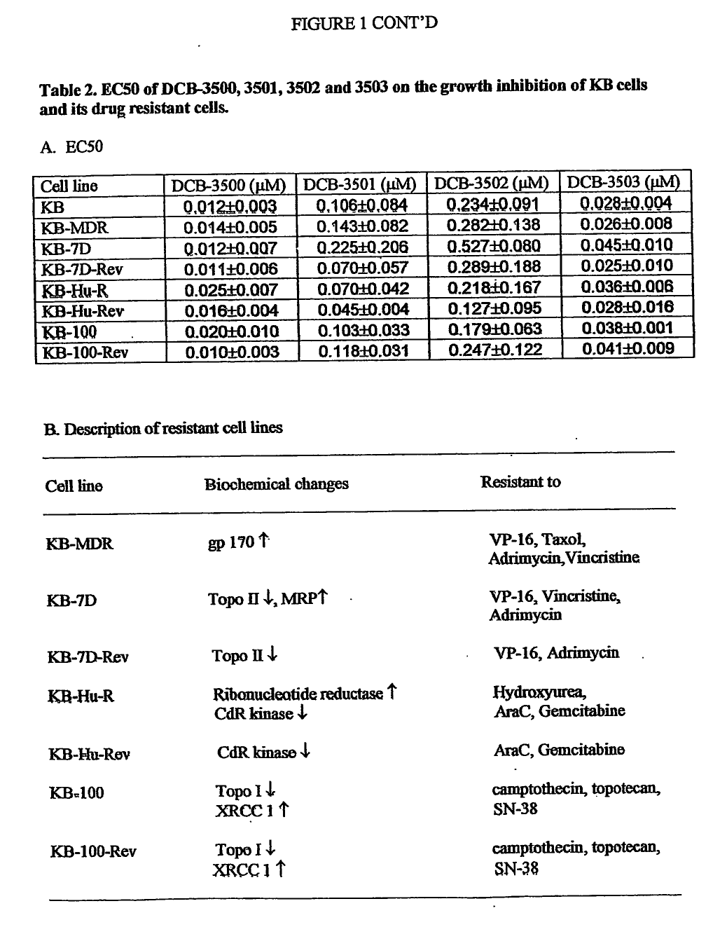

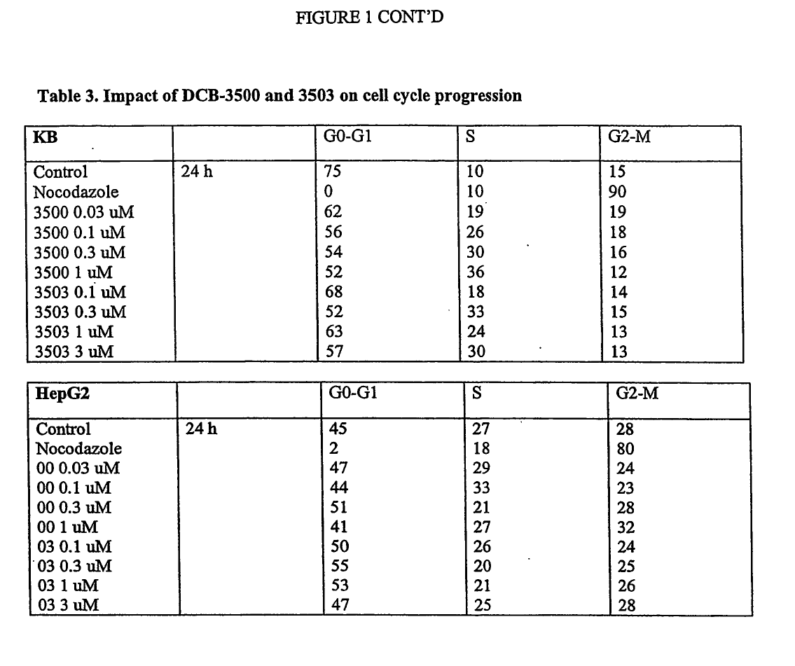

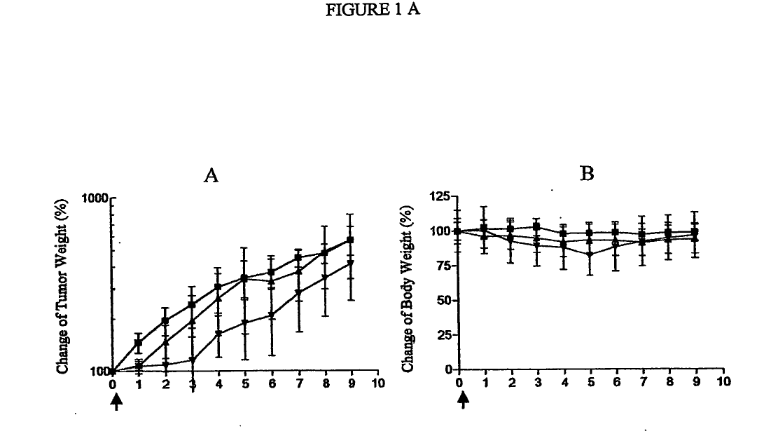

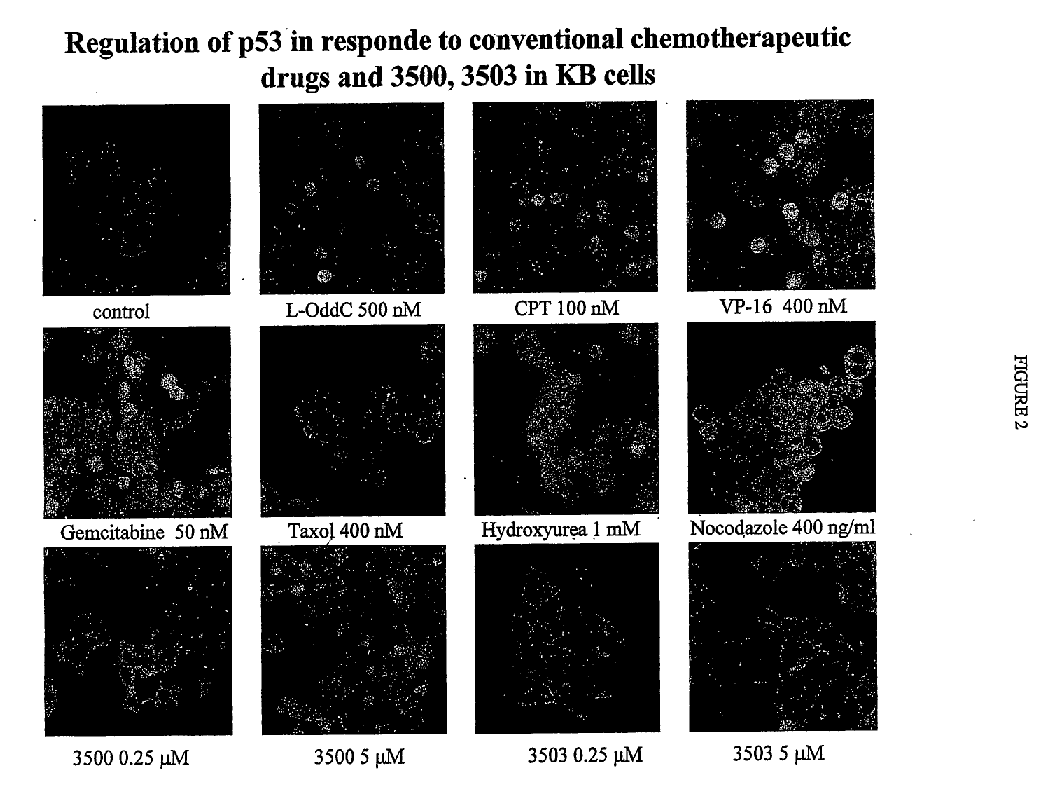

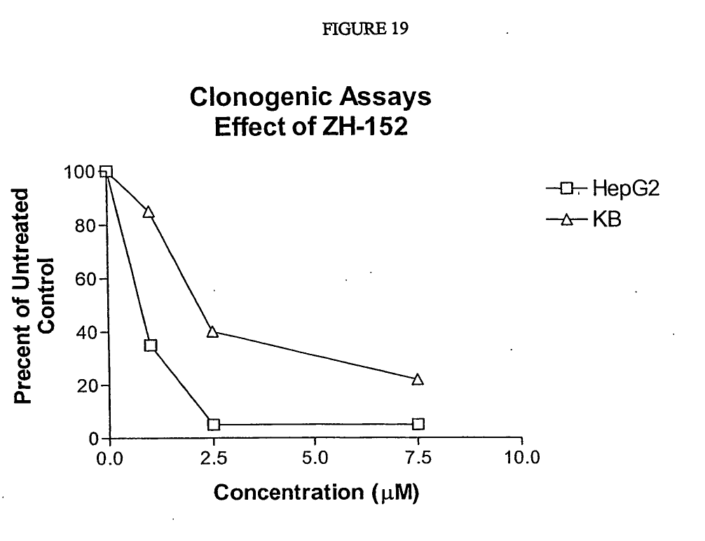

As shown in FIG. 19, KB and HepG2 cells were utilized to determine their clonogenic efficiency. The cell lines were exposed to II-3 for 24 hours at the concentrations indicated in FIG. 2. They were then grown in the absence of the drug. After eight generations, the colonies were stained and counted. The HepG2 cells were more sensitive to II-3 than were the KB cells.

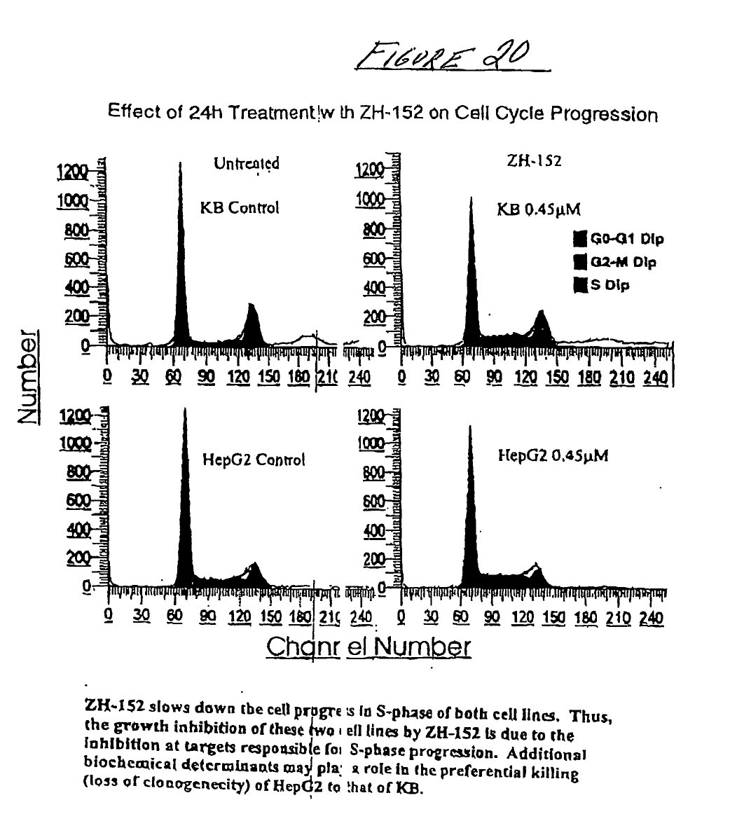

As shown in FIG. 20, using KB and HepG2 cell lines, compound II-3 (1×5 days, ip) demonstrated cell-growth suppression. The growth inhibition was due to inhibition at targets that are responsible for S-phase progression, which phase is involved with DNA replication. The preferential killing of HepG2 cells to KB cells suggests that different biochemical determinants are involved in these two cell lines.

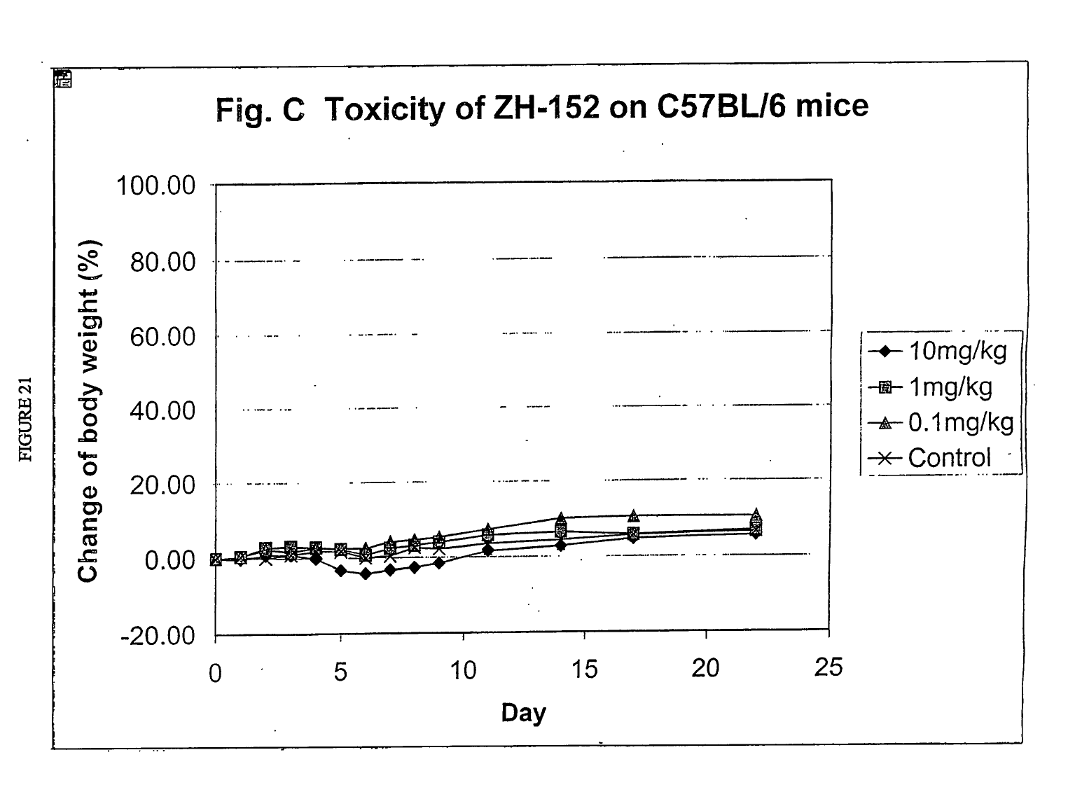

As shown in FIG. 21, using C57B1/6 mice, it was established that acute toxicity to compound II-3 is manageable and that 10 mg/kg dosing for 10 may be used for antitumor activity and further studies.

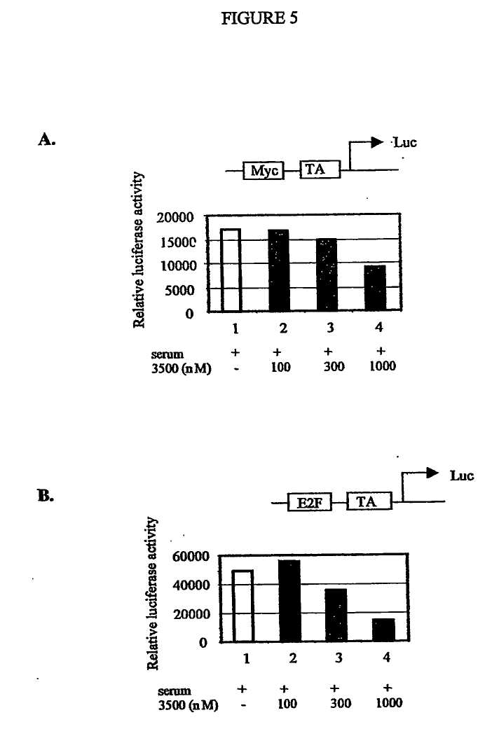

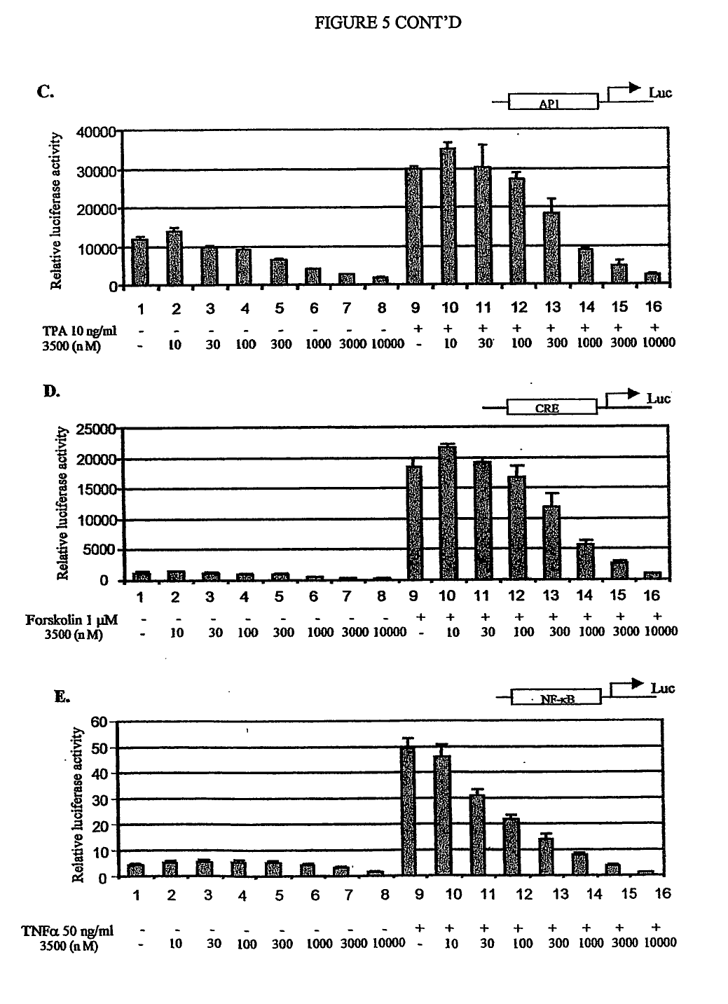

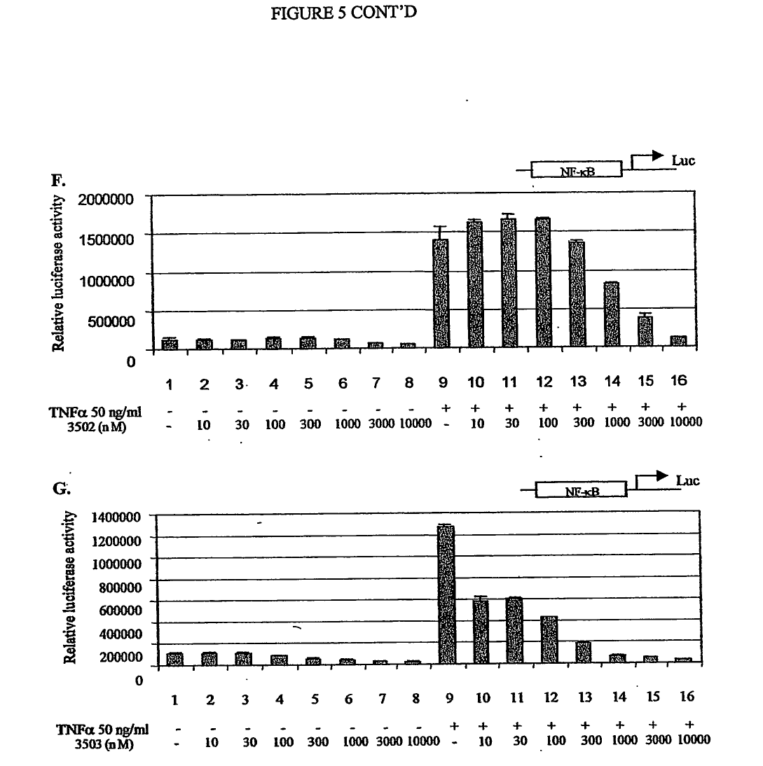

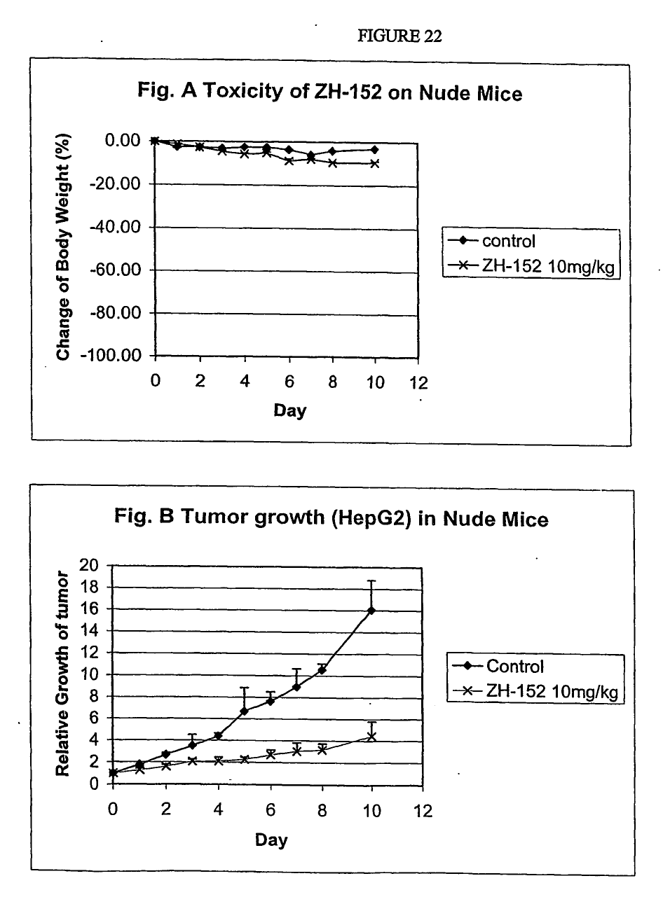

As shown in FIG. 22, compound II-3 was administered at 10 mg/kg ip once daily for 5 days to tumor-bearing (HepG2) mice. The weight loss (shown in FIG. 5A) and tumor growth (shown in FIG. 5B) were monitored. A profound antitumor effect without significant weight loss was demonstrated.

The potency of compound II-2 was found to be 3 to 5 times more than that of compound II-3 against HepG2 and KB cell growth in culture. This demonstrates that the OH group is not necessary for potent antitumor activity. The non-necessity of the OH group is potentially advantageous for purposes of chemical synthesis and chemical stability.

In Vivo Studies

(a) Antitumor Activity in Nude Mouse Bearing Human Tumor Model The human melanoma cell lines SK-MEL-2 and SK-MEL-28 (10⁶ cells) are implanted subcutaneously (s.c.) into the flank of six-week-old NCr Nude male mice (Taconic, Germantown, N.Y.). The drug-treatment experiment started when the tumor reaches a mass of approximately 100 mgs as determined by the formula Length−Width²/2. Tylo F and tylo G are given to a group of at least 5 mice at the concentrations determined by the toxicity testing, and different dosages of the drug is given once per day for five days. The tumor mass is calculated every second day, and if the tumor weight exceeds 2 g or more than 10% of the mouse body weight, the animal is euthanized by cervical dislocation. The tumored animals are observed for 45 days if the tumor is surpressed. Initially three dosages with a difference of 5-fold between each dose are given intraperitoneally (i.p.). The dose is adjusted (up or down) depending on the antitumor activity and the lethality caused by the drugs. LD₁₀ is the highest dose used. Once the antitumor activity of the drug i.p. has been established, an oral dose (p.o.) is also given the antitumor effect and oral bioavailability are examined. When SK-MEL-2 (sensitive) and SK-MEL-28 (resistant) are used, these two melanoma cell lines have different responses to tylo F and tylo G.

(b) Toxicity In the course of evaluating the antitumor activity of tylo F and tylo G in nude mice, the toxicity as manifested by body weight loss (every two days) and by blood abnormalities (every four days) is also monitored. When the blood is to be tested, a 20-μL sample of heparinized blood is taken from the retro-orbital plexus with a capillary tube. It is added to 200 μL of normal saline and analyzed on a BC 9100 hematology analyzer (Biochem Immune System, Allentown, Pa.). This allows monitoring of white blood cells, red blood cells and platelets, as well as the hematocrit, hemoglobin, mean corpuscular volume, mean corpuscular hemoglobin and platelet volume in these mice. In the event that there is animal death due to unexplained toxicity, tissues such as intestine, liver, kidney, lung, heart, brain and bone marrow is fixed in 10% formalin. The paraffin sections are examined by animal pathologists.

(c) Metabolism and Pharmacodynamic Study of the Compound of Interest The metabolism and pharmacodynamics of tylo F and tylo G are studied in tumor-bearing nude mice. Radioactive tylo F and tylo G are administered either i.p. or orally. The heparinized blood (200 μL) is collected from the retro-orbital plexus 5, 15, 30 min 1, 2, 4, 8, and 24 h, after drug injection. After centrifugation in microfuge tubes, the plasma supernatant is moved to a clean tube. Two parts of 100% methanol are added to each plasma sample, and they are incubated on ice for 15 min. After centrifugation for 5 min in a microfuge at approximately 15,000 rpm, the supernatant is moved to clean tube and stored at 70 □C until HPLC analysis as described above. The radioactivity from each sample is monitored using the in-line Packard Radiomatic Flow Scintillation Analyzer (Packard Instrument Co., Downers Grove, Ill.). In addition, the tylo metabolites in tumor and several major organs, such as the liver, intestine, lung, kidney, brain and bone marrow are also monitored 4, 8, 16 and 24 h following the treatment in a similar fashion. The structural identity of the radiolabelled tylo metabolites is analyzed. The WINLIN software package is used to determine the pharmacokinetic parameters of tylo F and tylo G, such as the T_{1/2}, area under curve, clearance and volume of distribution.

(d) Optimization of Treatment Protocol Based on the pharmacodynamics of tylo F and tylo G and the results of the antitumor activity studies using different routes of administration and different treatment schedules, the dosage and schedule of any given compound is altered to obtain maximal antitumor activity with the least toxicity.

The invention is described further in the following examples, which are illustrative only and in no way limiting.

### EXAMPLE 1

Materials and Methods

Materials

Cell culture media, fetal bovine serum (FBS) were purchased from Life Technologies. FuGENE 6 transfection reagent was from Roche. Standard chemotherapeutic agents, VP-16, Taxol, Hydroxyurea, Nocodazole, Gemcitabine, Camptothecine and others, Forskolin, 12-O-tetradecanoylphorbol 13-acetate (TPA), TNFα were purchased from Sigma-Aldrich (St. Louis, Mo.) and Calbiochem (San Diego, Calif.)

Plasmids

Firefly luciferase reporter vectors pMyc-TA-luc, pE2F-TA-luc, pAP1-luc, pCRE-luc were purchased from Clontech, Mercury™ pathway profiling system. pBIIX-luc were kindly provided by Dr. Ghosh (Yale University). Renilla luciferase reporter vector phRL was purchased from Promega.

Cell Culture

The human hepatocyte carcinoma cell line, HepG2, and the human nasopharyngeal carcinoma KB cells were maintained in RPMI 1640 medium supplemented with 10% fetal bovine serum (FBS). KB resistant cell lines, KB-MDR, KB-7D, KB-7D-Rev, KB-Hu-R, KB-Hu-Rev, KB-100, KB-100-Rev are described in FIG. 1, Table 2B.

### EXAMPLE 2

Cytotoxicity Assay

Cells (1×10⁴/well) were plated in 24-well plates. After 24 h, cells were treated with drugs for three generation times, then fixed and stained with 0.5% methylene blue in 50% ethanol for 2 h, followed by washing with tap water to remove excess color. Plates were dried and then resuspended in 1% sarkosyl, rotated at room temperature for 3 h. Cell growth was quantitated from the amount of methylene blue absorbed to the cells as measured by a spectrophotometer (Molecular Dynamics) at 595 nm. All experiments were performed in triplicate wells and were repeated at least three times. (see FIG. 1, Table 1B and Table 1C).

Clonogenic Assay

Cells (5×10⁴/well) were plated in 6-well plates. After 24 h, cells were treated with drugs for an additional 24 h. Cells were then trypsinized, counted, and cell viability was determined by trypan blue staining, 200 trypan blue negative cells were plated in triplicate in 6-well plates and grown for eight to ten generation times, then fixed and stained with 0.5% methylene blue in 50% ethanol for 1 h, after plates were washed and dried, the colonies were counted. (FIG. 1, Table 1C).

### EXAMPLE 3

Animal Studies

Four-week-old male NCR-nude mice were obtained from Taconic, and acclimated to laboratory conditions 1 week before tumor implantation. Human HepG2 tumor xenografts were established by injecting subcutaneously 2×10⁶ HepG2 cells. After 10 days, treatment was carried out I.P. by injecting 3 dosages of DCB-3500 and DCB-3503 at 30 mg/kg in every 8 hours on day 11 after tumor implanted. Tumor weight was estimated by using the equation: Length of tumor×(wide of tumor/2)². (FIG. 1(a))

Cell Cycle Analysis

KB and HepG2 cells were treated for 24 h with increasing concentrations of DCB-3500 and DCB-3503. At the end of treatment, cells were trypsinized, and the resulting cell suspensions were centrifuged at 1000 rpm for 5 min. The cells were fixed overnight in 70% ethanol at 4° C., centrifuged at 1000 rpm for 5 min, the pellets were washed twice with ice-cold PBS. Cell pellets were then resuspended in 0.5 ml PBS containing 50 μg/ml propidium iodide (Sigma-Aldrich) and 100 μg/ml RNase A (Sigma-Aldrich), incubated at 37° C. for 30 min, and then analyzed by FACScan using Cell Quest software (Becton Dickinson Labware, Franklin Lakes, N.J.). Data were analyzed using Modfit LT version 3.1 software (Verity Software House, Topsham, Me.) for cell cycle profile. (FIG. 1, Table 3)

Apoptosis Assay

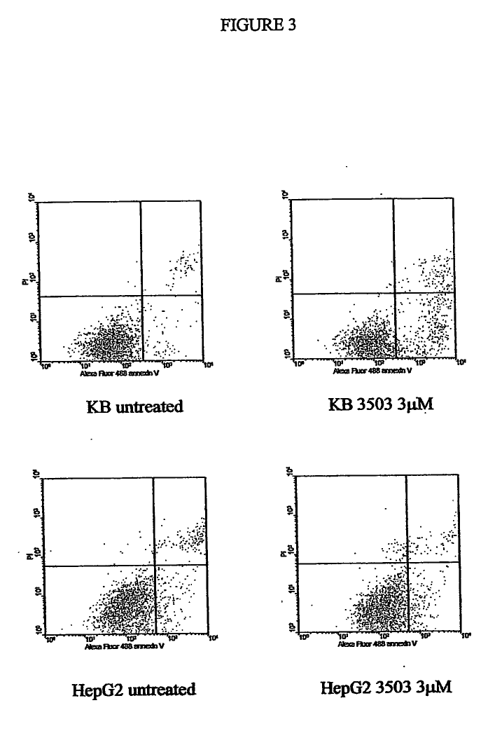

Apoptosis was determined by using Vybrant™ apoptosis assay kit (V-13241, Molecular Probes, Eugene, Oreg.) according to the manufacturer's instructions. Briefly, 1×10⁶ control or treated cellls were resuspended in annexin-binding buffer, then stained with Alexa Fluor 488 annexin V and propidium iodide, and then incubated at room temperature for 15 min. Stained cells were analyzed by flow cytometer (Becton Dickson, Franklin Lakes, N.J.). The population separated into three groups: live cells show a low level fluorescence, apoptotic cells show green fluorescence, necrotic cells show both red and green fluorescence. Data were analyzed using WinMDI version 2.8 software. See FIG. 3

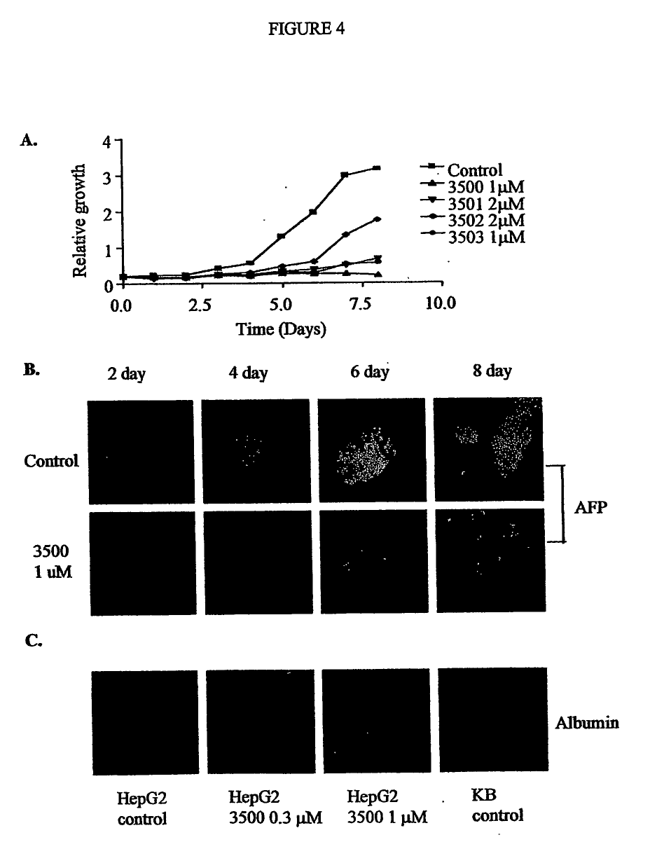

Cell growth inhibition for 24 h, and then monitor the cell growth in the absence of drug Cells (1×10⁴/well) were plated in 6-well plates. After 24 h, cells were treated with drugs for an additional 24 h. The drug-containing media was then removed, and the cells were incubated in drug-free media for another 1 to 8 days. At the end of each incubation period, cells were fixed and stained with 0.5% methylene blue in 50% ethanol and resuspended in 1% sarkosyl. The cell growth was determined as previously described in cytotoxicity assay. See FIG. 4.

Confocal Microscopy

The confocal microscopic analysis was performed using methods similar to those described previously. Briefly, 5×10⁴ HepG2 and KB cells were plated onto 22 mm×22 mm glass coverslips in 35-mm cluture dishes. After 24 h, cells were treated as indicated. At the end of incubation, cells were fixed with 4% paraformaldehyde at room temperature for 30 min, permeabilized by 0.5% Triton X-100 in PBS at room temperature for 15 min, then incubated with 3% BSA in PBS at 4° C. overnight to block non-specific binding. Cells were further incubated with p53 antibody (1:100), AFP antibody (1:100) or albumin antibody (1:100) at room temperature for 1 h, followed by FITC-conjugated anti-rabbit or anti-mouse antibody. Cells were sealed in antifade reagent (Molecular Probes). Confocal micrographs were scanned by laser scan confocal microscope, LSM 510 (Zeiss). See FIGS. 2 and 4.

Transfection and Luciferase Assay

HepG2 cells were plated at a density of 2×10⁴ per well (48-well plate) and transfected with 0.2 μg of firefly luciferase reporter vector pMyc-TA-luc, pE2F-TA-luc, pAP1-luc, pCRE-luc, or pBIIX-luc (containing two tandemly repeated NF-κB binding sites) respectively, along with internal control vector promoter-less renilla luciferase reporter vector phRL (Promega), using FuGENE 6™ transfection reagent according to the manufacturer's instructions. After 20 h, medium was changed, cells were then treated as indicated in the figure legends. Cell extracts were prepared and luciferase activity was measured using a Dual-luciferase (firefly and renilla luciferase) assay kit according to the manufacturer's instructions. See FIGS. 5A.-G.

1. A compound of the formula: 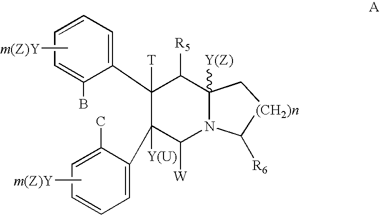
Wherein Y is O, S, NH, CH₂ or is absent;
Each (Z) is independently H, a (C₁-C₄) alkyl, a substituted alkyl, an aryl, a substituted aryl, alkyl silyl, a heterocycle, a substituted heterocycle, with the proviso that not all Z are H when Y is absent;
(U) is H, a (C₁-C₄) alkyl, a substituted alkyl, an aryl, a substituted aryl, alkyl silyl, a heterocycle, a substituted heterocycle, or together with W forms a double bond in the nitrogen containing ring or together with T forms a double bond in the nitrogen containing ring;
T is H, forms a double bond with the carbon to which R₅ is attached or forms a double bond with the carbon attached to Y(U);
W is H or forms a double bond with the carbon attached to Y(U) in the nitrogen containing ring;
R₅ is H, OH, ═O (to form a carbonyl group with the carbon to which it is attached), a carboxyl (carboxylate group), —OC(O)Rₓ group, a —C(O)Rₓ, or a —C(O)ORₓ group, where Rₓ is a C₂ to C₁₅ alkyl, preferably a C₂ to C₈ alkyl;
R₆ is H, OH, ═O (to form a carbonyl with the carbon to which it is attached), a carboxyl (carboxylate group), a —OC(O)Rₓ group, a —C(O)Rₓ, or a —C(O)ORₓ group, where Rₓ is defined above;
B is Y(Z) or together with C forms a bond between the two phenyl rings to which each of B and C is attached;
C is Y(Z) or together with B forms a bond between the two phenyl rings to which each of B and C is attached;
m is from 0 to 4;
n is from 0 to 3;
and epimers, pharmaceutically acceptable salts, solvates or polymorphs thereof.

2. A compound according to claim 1 of the formula: 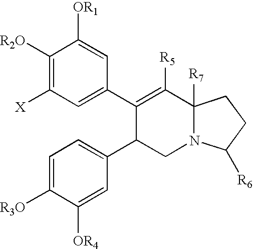
and the epimers, pharmaceutically acceptable salts, solvates, or polymorphs thereof, wherein R₁, R₂, R₃, R₄ and R₇ are the same or different and are either H, an alkyl, a substituted alkyl, an aryl, a substituted aryl, a heterocycle, or a substituted heterocycle;
R₅ is H, OH, a —OC(O)Rₓ group, a —C(O)Rₓ, or a —C(O)ORₓ group, where Rₓ is a C₂ to C₁₅ alkyl;
R₆ is H, a ═O group, a carboxyl (carboxylate group), a —OC(O)Rₓ group, a —C(O)Rₓ, or a —C(O)ORₓ group, where Rₓ is defined above;
X is H or is OR, where R is either H, an alkyl, a substituted alkyl, an aryl, a substituted aryl, a heterocycle, or a substituted heterocycle.

3. A compound of claim 1, wherein the compound has the formula: 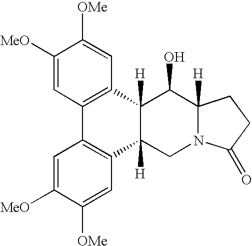
and the epimers, pharmaceutically acceptable salts, solvates, or polymorphs thereof.

4. A compound of claim 1, wherein the compound has the formula: 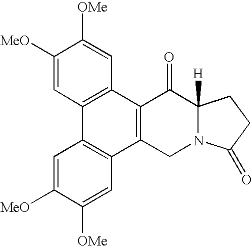
and its enantiomeric analogues, pharmaceutically acceptable salts, solvates, or polymorphs thereof.

5. A compound of claim 1, wherein the compound has the formula: 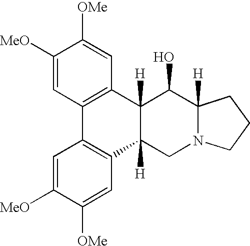
and its epimeric and enantiomeric analogues, pharmaceutically acceptable salts, solvates, or polymorphs thereof.

6. A compound of claim 1, wherein the compound has the formula: 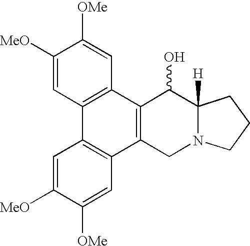
and its epimeric and enantiomeric analogues, pharmaceutically acceptable salts, solvates, or polymorphs thereof

7. A compound of claim 1, wherein the compound has the formula: 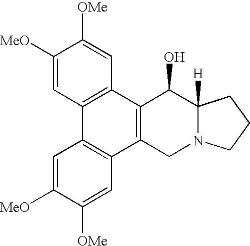
and its enantiomeric analogues, pharmaceutically acceptable salts, solvates, or polymorphs thereof.

8. A compound of claim 1, wherein the compound has the formula: 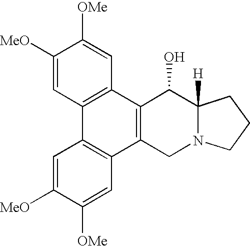
and its enantiomeric analogues, pharmaceutically acceptable salts, solvates, or polymorphs thereof.

9. A compound of claim 1, wherein the compound has the formula: 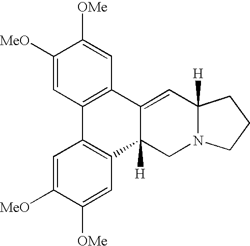
and its epimeric and enantiomeric analogues, pharmaceutically acceptable salts, solvates, or polymorphs thereof.

10. A compound of claim 1, wherein the compound has the formula: 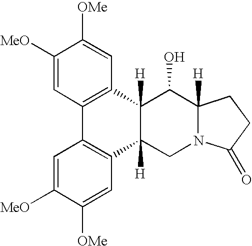
and its epimeric and enantiomeric analogues, pharmaceutically acceptable salts, solvates, or polymorphs thereof.

11. A compound of claim 1, wherein the compound has the formula: 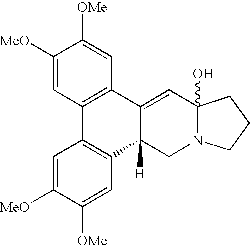
and its enantiomeric analogues, pharmaceutically acceptable salts, solvates, or polymorphs thereof.

12-16. (canceled)

17. A process of making a tyloindicine analogue comprising:
(a) effecting a Martin sulfurane dehydration of an alcohol of the formula 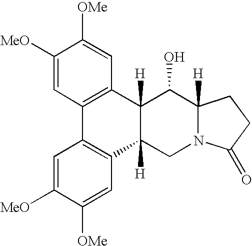
to yield an alkene of the formula 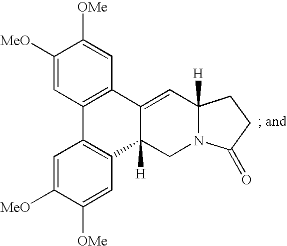
(b) reducing the alkene of step (a) in a reducing reaction medium to yield a tyloindicine analogue of the formula

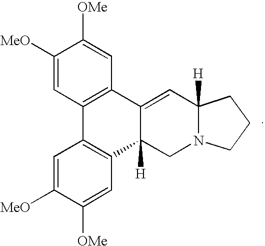

18-25. (canceled)

26. A pharmaceutical composition comprising a pharmaceutically acceptable carrier and a therapeutically effective amount of a compound of claim 1 or 2.

27. A pharmaceutical composition, comprising a pharmaceutically acceptable carrier and a therapeutically effective amount of a compound of claims 3-15 or 55-58.

28. A method of treating a mammal suffering from a neoplasia, comprising administering to the mammal in need thereof a therapeutically effective amount of one or more compounds of claims 1 or 2.

29. (canceled)

30. A method of treating a mammal suffering from cancer, comprising administering to the mammal in need thereof a therapeutically effective amount of one or more compounds of claims 1 or 2.

31. (canceled)

32. The method of claim 30 or 31, wherein the cancer is one or more of the following types: stomach, colon, rectal, liver, pancreatic, lung, breast, cervix uteri, corpus uteri, ovary, prostate, testis, bladder, renal, brain or central nervous system, head and neck, throat, Hodgkins disease, non-Hodgkins leukemia, multiple myeloma leukemias, skin melanoma, acute lymphocytic leukemia, acute mylogenous leukemia, Ewings Sarcoma, small cell lung cancer, choriocarcinoma, rhabdomyosarcoma, Wilms Tumor, neuroblastoma, hairy cell leukemia, mouth/pharynx, oesophagus, larynx, melanoma, kidney and lymphoma.

33. The method of claim 30, wherein the cancer is a drug resistant cancer.

34. The method of claim 33 wherein said cancer is resistant to at least one drug selected from the group consisting of alkylating agents, DNA-interactive compounds and topoisomerase-active agents.

35. The method according to claim 33 wherein said cancer is resistant to at least one drug selected from the group consisting of etoposide, gemcitabine, hydroxyurea, Topo I drugs and Topo II drugs.

36. The method of claim 28, wherein a compound of claim 1 or 2 are administered to inhibit growth of a neoplasia.

37. (canceled)

38. The method of claim 28, wherein the compound of claim 1 is coadministered with one or compounds selected from the group consisting of etoposide, cis-platin, carboplatin, lobaplatin, ormaplatin, oxaplatin, hexamethylmalamine, NLCQ-1, mephalan, dihydroxybusulfan, cyclophosphamide, daunorubicin, doxorubicin, mitomycin, adriamycin, camptothecin, vincristine, vinblastine, hydroxyurea, gemcitabine, Topo-I and Topo II drugs, polynucleotides, oligonucleotides, taxol, methacycline, anti-angiogenesis agents, azaindole derivatives, dibenzofluorene derivatives, temozolomide, AP/AMP and their prodrug forms.

39. The method of claim 28, wherein the neoplasia is a benign tumor.

40. The method according to 30, wherein the cancer is a malignant tumor.

41. The method according to claim 30, wherein the cancer has developed drug resistance.

42. The method according to claim 30, wherein the cancer is multiple drug resistant breast cancer.

43-44. (canceled)

45. The method according to claim 28, wherein the mammal is a human.

46. A method of treating a mammal suffering from an inflammatory or autoimmune disorder, comprising administering to the mammal in need thereof a therapeutically effective amount of one or more compounds of claim 1 or 2 or epimers, pharmaceutically acceptable salts, solvates, or polymorphs thereof.

47. (canceled)

48. The method of claim 46, wherein the inflammatory or autoimmune disorder is associated with the activation of NF-κB.

49. The method of claim 46 wherein said inflammatory or autoimmune disorder is a transplantation rejection, transplantation-associated vasculopathy, acute glomerulonephritis, lupus nephritis and tubulointerstitial nephritis, asthma, respiratory distress syndrome, gastritis, rheumatoid arthritis, lupus erythematosis), vasculitis, diabetes, AIDS, sepsis, thrombosis, coronary artery disease, restenosis after angioplasty or by-pass surgery, ischemia).

50. The method of claim 46 wherein said inflammatory or autoimmune disorder is rheumatoid arthritis, inflammatory bowel disease, asthma, dermatitis, psoriasis and atopic dermatitis, autoimmune diseases, tissue and organ rejection, Alzheimers disease, Hodgkin's disease, ADS and Ataxia Telangiestasia.

51. A method of treating an EBV infection comprising administering to a patient in need of therapy an effective amount of a compound according to claim 1 to said patient.

52. A method of treating EBV-related lymphoma or cancer in a patient comprising administering to a patient in need of therapy an effective amount of a compound according to claim 1 to said patient.

53-54. (canceled)

55. A compound of the formula: 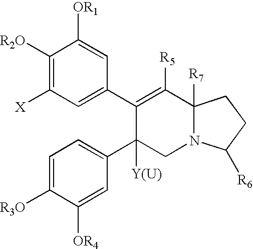
and the epimers, pharmaceutically acceptable salts, solvates, or polymorphs thereof, wherein R₁, R₂, R₃, R₄ and R₇ are the same or different and are either H, an alkyl, a substituted alkyl, an aryl, a substituted aryl, an alkyl silyl, a heterocycle, or a substituted heterocycle;
wherein Y is O, S, NH, CH₂ or is absent;
U is H, a (C₁-C₄) alkyl, a substituted alkyl, an aryl, a substituted aryl, alkyl silyl, a heterocycle, a substituted heterocycle, or together with W forms a double bond in the nitrogen containing ring;
R₅ is H, OH, O (to form a carbonyl group with the carbon to which it is attached), a —OC(O)Rₓ group, a —C(O)Rₓ, or a —C(O)ORₓ group, where Rₓ is a C₂ to C₁₅ alkyl, preferably a C₂ to C₈ alkyl;
R₆ is H, a carboxyl (carboxylate group), a —OC(O)Rₓ group, a —C(O)Rₓ, or a —C(O)ORₓ group, where Rₓ is defined above;
X is H or is OR_{b}, where R_{b} is either H, an alkyl, a substituted alkyl, an aryl, a substituted aryl, a heterocycle, or a substituted heterocycle.

56. A compound of claim 55, wherein the compound has the formula: 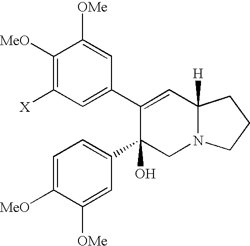
where X is H, OH, O(C₁-C₄) alkyl, O-benzyl, trialkylsilyl-O or diarylalkylsilyl-O and the epimers, pharmaceutically acceptable salts, solvates, or polymorphs thereof.

57. A compound of claim 55, wherein the compound has the formula: 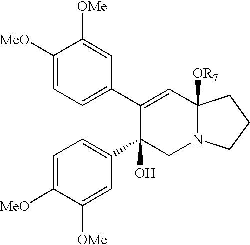
where R₇ is H, SiMe₃, or is 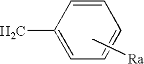
where Rₐ is either H, an alkyl, a substituted alkyl, an aryl, a substituted aryl, a heterocycle, or a substituted heterocycle, and the epimers, pharmaceutically acceptable salts, solvates, or polymorphs thereof.

58. A compound of claim 55, wherein the compound has the formula: 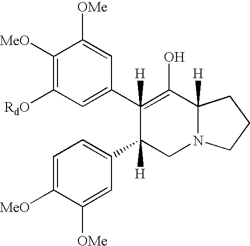
and the epimers, pharmaceutically acceptable salts, solvates, or polymorphs thereof, where R_{d} is H or a C₁-C₄ alkyl group.

59-62. (canceled)
